(19) United States
(12) Patent Application Publication
(10) Pub. No.: US 2021/0217493 A1
(43) Pub. Date: Jul. 15, 2021

(54) REDUCING NOISE IN SEQUENCING DATA

(71) Applicant: SeekIn, Inc., Lewes, DE (US)

(72) Inventors: Feng Zhang, Brookline, MA (US); Mao Mao, San Diego, CA (US); Hao Chen, Jersey City, NJ (US)

(21) Appl. No.: 17/263,340

(22) PCT Filed: Jul. 26, 2019
(86) PCT No.: PCT/US19/43704

§ 371 Date: Jan. 26, 2021

Related U.S. Application Data
(60) Provisional application No. 62/711,219, filed on Jul. 27, 2018.

(51) Int. Cl. G16B 30/20; C12Q 1/6869; G06F 17/18

(52) U.S. Cl. CPC G16B 30/20; G06F 17/18; C12Q 1/6869

(57) ABSTRACT

This disclosure is related to methods and apparatus of processing sequencing data (e.g., reducing noise in sequencing data).

### CLAIM OF PRIORITY

This application claims the benefit of U.S. Provisional Application No. 62/711,219, filed on Jul. 27, 2018. The entire contents of the foregoing are incorporated herein by reference.

### TECHNICAL FIELD

This disclosure is related to methods of processing sequencing data.

### BACKGROUND

In recent years, advancement in next generation sequencing has made detection of mutations in various types of biosamples possible on a genome-wide scale. However, it is still challenging to detect variants with low frequency, such as rare variants from tumor cells and circulating tumor DNA (ctDNA). The accuracy of calling rare variants is largely compromised by background noise in sequencing data. In order to improve rare variant calling accuracy, sequencing in greater depth has been proposed, but sequencing in greater generates a large amount of data and it is not suitable for clinic use because of its cost. In addition, it might be difficult to do deep sequencing if the sample is limited. There is a need to improve methods of processing sequence data, particularly reducing noise in sequencing data.

### SUMMARY

This disclosure is related to methods of reducing sequencing noise and/or detect rare variants. In some embodiments, the methods described herein can distinguish the signals for rare mutations from noise.

In one aspect, this disclosure provides methods for cancelling noise in sequencing results. The methods can involve one or more of the following steps:
(a) determining frequencies for each base type in control samples collected from a group of control subjects and determining frequencies for each base type in a sample collected from a subject having a tumor or suspected to have a tumor at a position of interest in the genome;
(b) determining a divergence score for the position of interest by calculating mutual entropy between the distribution of base type frequencies in control samples and the distribution of base type frequencies in the sample collected from the subject having a tumor or suspected to have a tumor;
(c) determining a significance score by determining that probability that the distribution of base type frequencies in control samples and the distribution of base type frequencies in the sample collected from the subject having a tumor or suspected to have a tumor represent the same distribution;
(d) calculating an information score based on the divergence score and the significance score, wherein a higher information score indicates that the sequencing results at the position of interest is more likely to be noise.

In some embodiments, the sample is derived from a biological sample, e.g., whole blood, plasma and tissues, or saliva. In some embodiments, the sample is circulating cell-free nucleic acids.

In some embodiments, the divergence score is calculated by the formula:

$${D}_{i} = \frac{1}{2} [ {{\sum }^{4}}_{j = 1} {}_{j}^{i}{Q}{}_{T}^{} {log}_{2} \frac{{}_{j}^{i}{Q}{}_{T}^{}}{{}_{j}^{i}{Q}{}_{v}^{}} + {{\sum }^{4}}_{j = 1} {}_{j}^{i}{Q}{}_{N}^{} {log}_{2} \frac{{}_{j}^{i}{Q}{}_{N}^{}}{{}_{j}^{i}{Q}{}_{v}^{}} ]$$

wherein ⁱⱼQ_{N} is the frequency for a base type j at position of interest i in the control sample, ⁱⱼQ_{T} is the frequency for a base type j at position i in the samples collected from a subject having a tumor or suspected to have a tumor.

In some embodiments,

$${}_{j}^{i}{Q}{}_{v}^{} = \frac{1}{2} ( {}_{j}^{i}{Q}{}_{T}^{} + {}_{j}^{i}{Q}{}_{N}^{} ) .$$

In some embodiments, the significance score is calculated by the formula:

$${S}_{i} = \frac{1}{2} [ {{\sum }^{4}}_{j = 1} {}_{j}^{i}{Q}{}_{v}^{} {log}_{2} \frac{{}_{j}^{i}{Q}{}_{v}^{}}{{}_{j}^{i} R} + {{\sum }^{4}}_{j = 1} {}_{j} p {log}_{2} \frac{{}_{j} p}{{}_{j}^{i} R} ]$$

In some embodiments, ⱼp is the background frequency of base j in a reference human genome.

In some embodiments,

$${}_{j}^{i} R = \frac{1}{2} {(}_{j}^{i} {Q}_{v} + {}_{j} p ) .$$

In some embodiments, the reference human genome is human genome assembly GRCh37 (hg19) or GRCh38(hg38).

In some embodiments, the information score is calculated by the formula:

$${I}_{i} = \frac{1}{2} ( 1 - {D}_{i} ) ( 1 + {S}_{i} ) .$$

In some embodiments, the sequencing results at the position of interest is removed if the information score is higher than a reference threshold.

In some embodiments, the sequencing results at the position of interest is included if the information score is lower than a reference threshold.

In one aspect, the disclosure also provides systems for cancelling noise in sequencing results comprising one or more of the following:
a) at least one device configured to sequence nucleic acid samples comprising a first group of nucleic acid samples collected from a group of control subjects and a second group of nucleic acid samples collected from a subject having a tumor or suspected to have a tumor;
b) a computer-readable program code comprising instructions to execute the following:
   i. calculating frequencies for each base type in the first group of samples and frequencies for each base type in the second group of samples at a position of interest in the genome;
   ii. calculating a divergence score for position of interest by calculating mutual entropy between the distribution of base type frequencies in the first group of samples and the distribution of base type frequencies in the second group of samples;
   iii. calculating a significance score by determining that probability that the distribution of base type frequencies in the first group of samples and the distribution of base type frequencies in the second group of samples represent the same distribution;
   iv. calculating an information score based on the divergence score and the significance score, wherein a higher information score indicates that sequencing results at the position of interest is more likely to be noise;
c) a computer-readable program code comprising instructions to execute the following:
   i. removing the sequencing results at the position of interest if the information score is higher than a reference threshold; or
   ii. including the sequencing results at the position of interest if the information score is lower than a reference threshold.

In another aspect, the disclosure also provides methods for cancelling noise in sequencing results. The methods involve one or more of the following steps:
(a) determining a ratio of frequencies of each base type in control samples collected from a group of control subjects to frequencies of each base type in a reference genome;
(b) determining a ratio of frequencies of each base type in a sample collected from a subject having a tumor or suspected to have a tumor as compared to frequencies of each base type in a reference genome;
(c) determining the product score for log of ratios of frequencies of each base type;
(d) removing the sequencing results if the product score is higher than a reference threshold.

In some embodiments, the log of the ratio of frequencies of each base type in samples collected from the subject having a tumor or suspected to have a tumor is determined by the following formula

$${}_{j}^{i}{w}{}_{T}^{} = ln \frac{{}_{j}^{i}{Q}{}_{T}^{}}{{}_{j} p}$$

wherein ⱼp is the background frequency of a base type j in a reference human genome, and ⁱⱼQ_{T} is the frequency for the base type j at position i in the sample collected from a subject having a tumor or suspected to have a tumor.

In some embodiments, the log of the ratio of frequencies of each base type in control samples is determined by the following formula

$${}_{j}^{i}{w}{}_{N}^{} = ln \frac{{}_{j}^{i}{Q}{}_{N}^{}}{{}_{j} p}$$

wherein ⱼp is the background frequency of a base type j in a reference human genome, and wherein ⁱⱼQ_{N} is the frequency for the base type j at position i in the control samples.

In some embodiments, the product score is determined by the following formula:

$${P}_{i} = {{\sum }^{4}}_{j = 1} {}_{j}^{i}{w}{}_{T}^{} {}_{j}^{i}{w}{}_{N}^{}$$

In some embodiments, the product score is determined by the following formula:

$${M}_{i} = {{\sum }^{4}}_{j = 1} ( {}_{j}^{i}{w}{}_{T}^{} + {}_{j}^{i}{w}{}_{N}^{} )$$

In one aspect, the disclosure provides a system for cancelling noise in sequencing data comprising:
a) at least one device configured to sequence nucleic acid samples comprising a first group of control nucleic acid samples and a second group of nucleic acid samples collected from a subject having a tumor or suspected to have a tumor;
b) a computer-readable program code comprising instructions to execute the following:
   i. determining a ratio of frequencies of each base type in the first group of control nucleic acid samples to frequencies of each base type in a reference genome;
   ii. determining a ratio of frequencies of each base type in the second group of nucleic acid samples to frequencies of each base type in a reference genome;
   iii. determining a score for log of ratios of frequencies of each base type; and
   iv. removing the sequencing results if the score has an absolute value that is higher than a reference threshold.

In one aspect, the disclosure provides a computer-implemented method of reducing noise in sequencing data, the method comprising:
a) receiving a plurality of sequence reads obtained from sequencing a group of case nucleic acid samples and a group of control nucleic acid samples;
b) aligning the plurality of sequence reads to a target region in a reference genome;
c) determining frequencies for each base type at a position of interest in the target region in the group of control samples;
d) determining frequencies for each base type at the position of interest in the target region in the group of case samples;
e) determining a divergence score for the position of interest by calculating mutual entropy between the distribution of base type frequencies in the group of control samples and the distribution of base type frequencies in the samples collected in the group of case samples;
f) determining a significance score by determining the likelihood that the distribution of base type frequencies in the group of control samples and the distribution of base type frequencies in the group of cases sample represent the same distribution; and
g) determining whether sequencing results at the position of interest is likely to be sequencing noise based on the divergence score and the significance score.

In some embodiments, the method further comprises:

h) calculating an information score based on the divergence score and the significance score;

i) reporting sequencing results at the position of interest if the information score for the position of interest is less than a reference threshold; and

j) removing sequencing results at the position of interest if the information score for the position of interest is higher than a reference threshold.

In some embodiments, the case samples and the control samples are derived from cell-free DNA fragments. In some embodiments, the case samples and the control samples are derived from RNA of a biological sample. In some embodiments, the case samples and the control samples are sequenced less than 1, 2, 3, 4, 5, 6, 7, 8, 9, 10, 15, or 20 fold.

In one aspect, the disclosure provides a computer-implemented method of reducing noise in sequencing data, the method comprising:
a) receiving a plurality of sequence reads obtained from sequencing a group of case nucleic acid samples and a group of control nucleic acid samples;
b) aligning the plurality of sequence reads to a target region in a reference genome;
c) determining a ratio of frequencies of each base type in control samples to frequencies of each base type in a reference genome;
d) determining a ratio of frequencies of each base type in case samples to frequencies of each base type in a reference genome;
e) determining a score for log of ratios of frequencies of each base type;
f) removing the sequencing results if the score has an absolute value that is higher than a reference threshold; or keeping the sequencing results if the score has an absolute value that is not greater than a reference threshold.

In one aspect, the disclosure provides a method for detecting DNA variation in a sample DNA sequence, comprising:
a) aligning sequence reads of the sample DNA sequences to a reference DNA sequence, thereby identifying a variant at a position of interest in the reference DNA sequence, and determining frequencies for each base type at the position of interest in the samples DNA sequences
b) determining frequencies for each base type at the position of interest in a group of control nucleic acid samples;
c) determining a divergence score for the position of interest by calculating mutual entropy between the distribution of base type frequencies in the samples and the distribution of base type frequencies in the control samples;
d) determining a significance score by determining the likelihood that the distribution of base type frequencies in the samples and the distribution of base type frequencies in the control sample represent the same distribution;
e) calculating an information score based on the divergence score and the significance score; and
   outputting the variant at the position of interest.

As used herein, the term “single nucleotide polymorphism” or “SNP” refers to the polynucleotide sequence variation present at a single nucleotide residue within different alleles of the same genomic sequence. This variation may occur within the coding region or non-coding region (i.e., in the promoter or intronic region) of a genomic sequence, if the genomic sequence is transcribed during protein production. Detection of one or more SNP allows differentiation of different alleles of a single genomic sequence or between two or more individuals. In some embodiments, the frequency of the SNP within a population is about or at least 1%, 2%, 3%, 4%, 5%, 6%, 7%, 8%, 9%, 10%, 15%, or 20%. In some embodiments, the frequency of the SNP within a population is less than 2%, 3%, 4%, 5%, 6%, 7%, 8%, 9%, 10%, 15%, or 20%.

As used herein, the term “a single-nucleotide variant” or “SNV” refers to a variation in a single nucleotide without any limitations of frequency. The SNV can arise in somatic cells.

As used herein, the term “allele” refers to one of several alternate forms of a gene or non-coding regions of DNA that occupy the same position on a chromosome. The term allele can be used to describe DNA from any organism including but not limited to bacteria, viruses, fungi, protozoa, molds, yeasts, plants, humans, non-humans, animals, and archeabacteria.

As used herein, the term “sample” refers to a specimen containing nucleic acid. Examples of samples include, but are not limited to, tissue, bodily fluid (for example, blood, serum, plasma, saliva, urine, tears, peritoneal fluid, ascitic fluid, vaginal secretion, breast fluid, breast milk, lymph fluid, cerebrospinal fluid or mucosa secretion), umbilical cord blood, chorionic villi, amniotic fluid, an embryo, embryonic tissues, lymph fluid, cerebrospinal fluid, mucosa secretion, or other body exudate, fecal matter, an individual cell or extract of the such sources that contain the nucleic acid of the same, and subcellular structures such as mitochondria, using protocols well established within the art.

As used herein, the term “sensitivity” refers to the proportion of true positives that are correctly identified as positives. It can be calculated by dividing the number of true positives by the number of true positives plus the number of false negatives.

As used herein, the term “specificity” refers to the proportion of true negatives that are correctly identified as negatives. It can be calculated by dividing the number of true negatives by the number of true negatives plus the number of false positives.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the methods described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

As used herein, the term “case sample” refers to a sample obtained from a subject who is at risk of having a disease or a disorder, is suspected of having a disease or a disorder, or has a disease or a disorder of interest. In some embodiments, the disease or disorder is cancer.

As used herein, the term “control sample” refers to a sample obtained from a subject who is healthy or does not have a disease or a disorder of interest (e.g., cancer).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. ROC plot of information score, Log Odds Product Score and Log Odds Sum Score.

FIG. 2A. Information scores for the top 200 mutation calls. The mutations are sorted by information scores.

FIG. 2B. Log Odds Product Score for the top 200 mutation calls. The mutations are sorted by the Log Odds Product Scores.

FIG. 2C. Log Odds Sum Score for the top 200 mutation calls. The mutations are sorted by the Log Odds Sum Scores.

FIG. 3A. Relationship between target allele frequency and information scores.

FIG. 3B. Relationship between target allele frequency and Log Odds Product Scores.

FIG. 3C. Relationship between target allele frequency and Log Odds Sum Score.

FIG. 4. Relationship between the observed allele frequency and the target allele frequency.

FIG. 5A shows the relationship between information score and the observed allele frequency.

FIG. 5B shows the relationship between Log Odds Product Score and the observed allele frequency.

FIG. 5C shows the relationship between Log Odds Sum Score and the observed allele frequency.

FIG. 6A. True positives among the mutations with top 200 information scores obtained from sequencing data with 500× coverage.

FIG. 6B. True positives among the mutations with top 200 information scores obtained from sequencing data with 200× coverage.

FIG. 6C. True positives among mutations with top 200 information scores obtained from sequencing data with 100× coverage.

FIG. 6D. True positives among mutations with top 200 information scores obtained from sequencing data with 50× coverage.

FIG. 6E. True positives among mutations with top 200 information scores obtained from sequencing data with 20× coverage.

FIG. 6F. True positives among mutations with top 200 information scores obtained from sequencing data with 10× coverage.

FIG. 6G. True positives among mutations with top 200 information scores obtained from sequencing data with 5× coverage.

FIG. 6H. True positives among mutations with top 200 information scores obtained from sequencing data with 2× coverage.

FIG. 7A. True positives among mutations with top 200 information scores obtained from ACRG Subject ID 200 (depth>20). 33 out of 33 true positives were detected. The last true positive ranks the 62^{nd}.

FIG. 7B. True positives among mutations with top 200 information scores obtained from ACRG Subject ID 11 (depth>20). 26 out of 27 true positives were detected. The last true positive ranks the 106^{nd}.

FIG. 7C. True positives among mutations with top 200 information scores obtained from ACRG Subject ID 22 (depth>20). 37 out of 37 true positives were detected. The last true positive ranks the 63rd.

FIG. 7D. True positives among mutations with top 200 information scores obtained from ACRG Subject ID 26 (depth>20). 69 out of 70 true positives were detected. The last true positive among the top 200 mutations ranks the 192nd.

FIG. 7E. True positives among mutations with top 200 information scores obtained from ACRG Subject ID 68 (depth>20). 10 out of 10 true positives were detected. The last true positive among the top 200 mutations ranks the 61st.

FIG. 7F. True positives among mutations with top 200 information scores obtained from ACRG Subject ID 82 (depth>20). 37 out of 37 true positives were detected. The last true positive among the top 200 mutations ranks the 108th.

FIG. 8 is a schematic diagram showing a system for detecting and minimizing sequencing noise.

### DETAILED DESCRIPTION

This disclosure is related to methods of reducing sequencing noise at each nucleotide position, methods for cancelling sequencing noise associated with technical origins, and methods of calling mutation based on probability that nucleotide is a mutation.

The methods are based on, in part, on the fact that the distribution of base frequencies (also known as nucleotide frequencies) in true mutation is statistically different from that in sequencing noises. Several scoring schemes are proposed herein to capture this subtle difference. These scores are designed to reflect the statistically significant difference of base frequency between true mutation and background noise. In some embodiments, every read is equally weighted and no normalization in performed since frequencies rather than base counts are used.

For these scores, nucleotide positions with true mutations are generally assigned lower scores (e.g., a lower absolute value of the score) and noise will have higher scores (e.g., a higher absolute value). Hence, a suitable score cutoff can be set so that, with a prospective false positive rate, nucleotide positions with their score below the cutoff can be confidently considered as true mutations, and nucleotide positions have a score above the cutoff (i.e. noise) can be detected and removed from further analysis.

The present disclosure provides a thorough characterization of sequencing data that can facilitate the detection of method-dependent systematic technical errors, and furthermore allow true variants to be accurately distinguished. The methods as described herein can determine sequencing noise/error at each nucleotide base position so that sequencing noise of technical origin can be cancelled. Thus, mutation can be called more accurately based on the well-calculated scores (e.g., probabilities).

### Sequencing and Sequencing Noise

Early diagnosis of cancer can generally increase the chances for successful treatment. Delays in accessing cancer care are common with late-stage presentation, particularly in lower resource settings and vulnerable populations. The consequences of delayed or inaccessible cancer care are lower likelihood of survival, greater morbidity of treatment and higher costs of care, resulting in avoidable deaths and disability from cancer. Early diagnosis improves cancer outcomes by providing care at the earliest possible stage and is therefore an important public health strategy in all settings.

Clinical use of cell free DNA (cfDNA) or circulating tumor DNA (ctDNA) analysis requires accurate assays for the genetic characterization of DNA fragments within the fluid of interest, e.g., blood. These assays often require high analytical sensitivity to detect clinically relevant genetic alterations in a high background of noise, e.g., wild-type DNA shed by nonmalignant cells. Low allelic frequencies (AF <0.5% mutant AF) are commonly seen in patients, particularly in the context of early detection. In addition, exquisite specificity is required because false positives can lead to further unnecessary, invasive testing or inappropriate treatment adjustment. Thus, it is important to distinguish true mutations (e.g., accurate variant calling) from sequencing noise. The present disclosure provides methods of reducing noise from sequencing data, particularly when the mutant allelic frequency is low.

DNA in samples are sequenced by the methods described herein, e.g., by Illumina platform (e.g. X-10, NovaSeq). In some embodiments, these samples are from control subjects, healthy subjects, tumor patients, patients who are at risk or suspected to have tumors. As used herein, the control subject can refer to a healthy subject, or a subject does not have a disease or a disorder of interest (e.g., cancer, tumor). The qualities of raw output reads can be checked by various quality control tools, e.g., FastQC. In some embodiments, the raw data are trimmed (e.g., by Fastp) to remove low-quality reads (e.g., any read having more than 40% of base quality less than 20 and/or any read shorter than 70 bp after all default trimming). In some embodiments, remaining data are checked by FastQC again to confirm that they still meet the quality criteria. Data passing quality control (QC) after trimming are aligned by an alignment tool, e.g., BWA (0.7.17-r1194-dirty).

The sequence reads can be aligned and mapped to a reference genome. The allele frequency at a particular position (e.g., in the reference genome) can be calculated. In order to determine whether a rare variant at this position is likely to be noise, quality scores can be calculated based on the methods described herein.

The methods are based on, in part, on the fact that the distribution of base frequencies (or nucleotide frequencies) in true mutation is statistically different from that in sequencing noises. In some embodiments, the quality score can be information score, Log Odds Product Score, or Log Odds Sum score. These scores are described herein and can be calculated from base frequency. Particularly, the information score as described herein can effectively reduce sequencing noise.

As used herein, the “base frequency” or “nucleotide frequency” at a position of interest refers to the frequency of the nucleotide in a group of nucleic acid samples. These nucleic acid samples can be from a subject (e.g., a control subject, a healthy subject, a subject who has tumors or cancers, a subject who is at risk of having tumors or cancers, a subject who is suspected to have tumors or cancers, or a subject who has some other disorders), or a group of subjects (e.g., control subjects, healthy subjects, subjects who have tumors or cancers, subjects who are at risk of having tumors or cancers, subjects who are suspected to have tumors or cancers, or subjects who have some other disorders). In some embodiments, the variant of interest is a somatic mutation (e.g., a mutation existing in a cancer cell). Thus, even if all nucleic acid samples are obtained from one subject, some nucleic acid samples (e.g., cfDNA or ctDNA) can have a variant that does not exist in the normal tissue samples of the same subject. Thus, in some embodiments, the base frequency or nucleotide frequency can be the frequency of a particular base or a nucleotide in cfDNA or ctDNA obtained from a subject. In some embodiments, the base frequency or nucleotide frequency can be the frequency of a particular base or a nucleotide in all cfDNA or ctDNA obtained from a group of subjects. In some embodiments, the variant has a frequency that is less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 20%, e.g., within the group of nucleic acids samples or sequence reads. In some embodiments, the variant has a frequency that is at least 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or 20%, e.g., within the group of nucleic acids samples or sequence reads. In some embodiments, the base frequency or nucleotide frequency in a reference genome is the frequency of the nucleotide in a population without considering somatic mutations or some other random mutations.

### Information Score

Given read alignments in the data file (e.g., BAM file), i is the position of interest on the genome and j is the base type (i.e., A, T, C, G) on this position. In some embodiments, the parameters from the samples collected from tumor patients or patients who are suspected to have tumors are designated as T (or tumor) and those from the normal samples (e.g., control samples, samples collected from subjects without tumors) are designated as N (or normal). Thus ⁱⱼQ_{T} is the observed frequency of a base type j at Position i in the samples collected from a tumor patient or a patient who is suspected to have tumor. In some embodiments, ⁱⱼQ_{T} is the observed frequency in the samples collected from one or more patients.

Similarly, ⁱⱼQ_{N} is the observed frequency in one or more normal samples or control samples. In some embodiments, ⁱⱼQ_{N} is the observed frequency in a group of nucleic acid samples obtained from one normal subject. In some embodiments, ⁱⱼQ_{N} is the observed frequency in a group of nucleic acid samples obtained from a group of normal subjects. Thus, in some cases, ⁱⱼQ_{N} can be the average of observed frequency within the group of normal subjects. The normal samples can be sequenced during the same time with the tumor samples. In some embodiments, the normal samples are not sequenced at the same time with the tumor samples. In some embodiments, ⁱⱼQ_{N} can be stored in a database. Thus, there is no need to repeatedly sequence normal samples.

The divergence score D at Position j is defined as:

$${D}_{i} = \frac{1}{2} [ {{\sum }^{4}}_{j = 1} {}_{j}^{i}{Q}{}_{T}^{} {log}_{2} \frac{{}_{j}^{i}{Q}{}_{T}^{}}{{}_{j}^{i}{Q}{}_{v}^{}} + {{\sum }^{4}}_{j = 1} {}_{j}^{i}{Q}{}_{N}^{} {log}_{2} \frac{{}_{j}^{i}{Q}{}_{N}^{}}{{}_{j}^{i}{Q}{}_{v}^{}} ]$$

wherein

$${}_{j}^{i}{Q}{}_{v}^{} = \frac{1}{2} ( {}_{j}^{i}{Q}{}_{T}^{} + {}_{j}^{i}{Q}{}_{N}^{} )$$

For a position i in the genome, if a given base type j has a frequency 0 at this position in both samples from normal subjects and samples from tumor patients or patients who are suspected to have tumors, that is both ⁱⱼQ_{T} and ⁱⱼQ_{N} are 0, a pseudo count frequency can be used in order to avoid the situation when the denominator (e.g., ⁱⱼQᵥ) is 0. In some embodiments, the pseudo count frequency is less than 0.001, 0.0009, 0.0008, 0.0007, 0.0006, 0.0005, 0.0004, 0.0003, 0.0002, or 0.0001. In some embodiments, the pseudo count frequency is at least or about 0.001, 0.0009, 0.0008, 0.0007, 0.0006, 0.0005, 0.0004, 0.0003, 0.0002, or 0.0001. In some embodiments, the pseudo count frequency is at least or about 0.00033. In some embodiments, the pseudo count frequency is applied only when the denominator is 0.

The divergence score indicates the mutual entropy between the distribution of base frequencies on true mutations and that on noises. In some embodiments, the noises are determined by the distribution of base frequencies in one or more control subjects (e.g., healthy subjects or subjects do not have cancers or tumors). In some embodiments, one subject is used to determine the base frequencies. In some embodiments, more than 1 subjects (e.g., about or more than 2, 3, 4, 5, 6, 7, 8, 9, 10, 20, 30, 40, 50, 60, 70, 80, 90, 100, 110, 120, 130, 140, 150, 160, 170, 180, 190, or 200) are used to determine the base frequencies. A large divergence score means the samples share less information and are not similar in terms of base frequencies.

An exemplary dataset is shown in Table 1 for illustration purpose. In Table 1, tumor samples and normal samples in Dataset 1 have quite different nucleotide frequencies so the divergence score is large. Nucleotide frequencies in Dataset 2 are more similar and thus the divergence score is much smaller than Dataset 1.

TABLE 1
Divergence Score Examples

| | Normal | | | | Tumor | | | | |
|---|---|---|---|---|---|---|---|---|---|
| | samples | | | | samples | | | | Divergence |
| Frequency | A | T | C | G | A | T | C | G | score |
| Dataset 1 | 94% | 2% | 2% | 2% | 28% | 1% | 70% | 1% | 0.1302 |
| Dataset 2 | 94% | 2% | 2% | 2% | 90% | 2% | 6% | 2% | 0.0024 |

The significance score S is defined as:

$${S}_{i} = \frac{1}{2} [ {{\sum }^{4}}_{j = 1} {}_{j}^{i}{Q}{}_{v}^{} {log}_{2} \frac{{}_{j}^{i}{Q}{}_{v}^{}}{{}_{j}^{i} R} + {{\sum }^{4}}_{j = 1} {}_{j} p {log}_{2} \frac{{}_{j} p}{{}_{j}^{i} R} ]$$

wherein

$${}_{j}^{i} R = \frac{1}{2} ( {}_{j}^{i}{Q}{}_{v}^{} + {}_{j} p )$$

ⱼp is the background frequency of base j in the whole human genome (e.g., the frequencies in hg19 or hg38 reference genomes). In some embodiments, it is the frequency in a relevant population (e.g., Caucasian, Asians, or black people).

The significance score estimates the probability of true mutation and noise actually representing the same source distribution. If the somatic mutation is false, its nucleotide frequencies will be a resampling from the underlying source distribution or the normal sample's distribution. Therefore, the significance score will be large if the mutation call is false.

Table 2 shows a dataset for illustration purpose. In Table 2, ⱼp is set to 0.25 for A, T, C and G, respectively.

TABLE 2
Significance score Examples

| | Normal | | | | Tumor | | | | Sig- |
|---|---|---|---|---|---|---|---|---|---|
| | samples | | | | samples | | | | nificance |
| Frequency | A | T | C | G | A | T | C | G | Score |
| True Mutation | 94% | 2% | 2% | 2% | 28% | 1% | 70% | 1% | 0.0738 |
| False Mutation | 94% | 2% | 2% | 2% | 90% | 2% | 6% | 2% | 0.1130 |

Based on above formula, in some embodiments, information score at Position i can be calculated based on the following equation:

$${I}_{i} = \frac{1}{2} ( 1 - {D}_{i} ) ( 1 + {S}_{i} )$$

In some embodiments, a small information score for the nucleotide position indicates a true mutation (rather than the noise) exists at this position in the tumor samples.

In some embodiments, an appropriate reference threshold can be used. In some embodiments, an information score of less than 0.4, 0.5, 0.6, 0.61, 0.62, 0.63, 0.64, 0.65, 0.66, 0.67, 0.68, 0.69, 0.7, or 0.8 is desirable. In some embodiments, a variant with an information score of about or at least 0.4, 0.5, 0.6, 0.61, 0.62, 0.63, 0.64, 0.65, 0.66, 0.67, 0.68, 0.69, 0.7, or 0.8 is treated as noise.

### Log Odds Product Score

In some embodiments, Log Odds Product Score can be used to assess the quality at the position.

The log odds of base type j at position of interest i in the tumor samples (T) and the normal (N) samples are defined as:

$${}_{j}^{i}{w}{}_{T}^{} = ln \frac{{}_{j}^{i}{Q}{}_{T}^{}}{{}_{j} p}$$

$${}_{j}^{i}{w}{}_{N}^{} = ln \frac{{}_{j}^{i}{Q}{}_{N}^{}}{{}_{j} p}$$

wherein ⱼp is the background frequency of base j in the whole human genome (e.g., the frequencies in hg19 or hg38 reference genomes). Similarly, for a particular base, if ⱼp is 0, a pseudo count frequency is used.

In some embodiments, the Log Odds Product Score at Position i can be calculated by the following equation:

$${P}_{i} = {\sum }_{j = 1}^{4} {}_{j}^{i}{w}{}_{T}^{} {}_{j}^{i}{w}{}_{N}^{}$$

It can be proven that only if ⁱⱼw_{T}=ⁱⱼw_{N}, can Log Odds Product Score reach the maximum. The larger difference between ⁱⱼw_{T} and ⁱⱼw_{N} is, the smaller Log Odds Product Score is. Table 3 shows an exemplary dataset for illustration purpose.

TABLE 3
Log Odds Product Score Examples

| | Normal | | | | Tumor | | | | |
|---|---|---|---|---|---|---|---|---|---|
| | samples | | | | samples | | | | |
| Frequency | A | T | C | G | A | T | C | G | Score |
| Dataset 1 | 94% | 2% | 2% | 2% | 28% | 1% | 70% | 1% | 2.6046 |
| Dataset 2 | 94% | 2% | 2% | 2% | 90% | 2% | 6% | 2% | 3.4063 |

A large Log Odds Product Score indicates that the sequencing results at this position are more likely to be a noise. Thus, if there is noise, the score will be higher. If there is a true mutation, the score will be lower.

In some embodiments, an appropriate reference threshold for Log Odds Product Score can be used. In some embodiments, a Log Odds Product Score of less than 80, 85, 90, 95, or 100 is desirable. In some embodiments, a variant with a Log Odds Product Score of about or at least 80, 85, 90, 95, or 100 is treated as noise.

### Log Odds Sum Score

In some embodiments, the Log Odds Sum Score can be used to assess the quality at the position. ⁱⱼw_{T} and ⁱⱼw_{T} can be calculated based on the equations described above.

In some embodiments, the Log Odds Sum Score at Position i can be calculated by the following equation:

$${M}_{i} = {\sum }_{j = 1}^{4} ( {}_{j}^{i}{w}{}_{T}^{} {+}_{j}^{i} {w}_{N} )$$

Because of the logarithm in the equation of calculating ⁱⱼw_{T} and ⁱⱼw_{T}, the Log Odds Sum Score is usually negative. In some embodiments, the absolute value of the Log Odds Sum Score can be used. A large absolute value indicates that the sequencing results at this position are more likely to be a noise. Thus, if there is noise, the absolute value will be higher. If there is a true mutation, the absolute value will be lower.

In some embodiments, an appropriate reference threshold for Log Odds Sum Score can be used. In some embodiments, a Log Odds Sum Score has an absolute value of less than 28, 29, 30, 31, 35, or 40 is desirable. In some embodiments, a variant with the absolute value of a Log Odds Sum Score of about or at least 28, 29, 30, 31, 35, or 40 is treated as noise.

### Evaluating Quality Score

The methods described herein can be evaluated for its ability to characterize sequencing noise. Various statistical criteria can be used, for example, area under the curve (AUC), percentage of correct predictions, sensitivity, and/or specificity. In one embodiment, the methods are evaluated by cross validation, Leave One OUT Cross Validation (LOOCV), n-fold cross validation, and jackknife analysis.

In some embodiments, the method used to evaluate the mathematical models is a method that evaluates the sensitivity (true positive fraction) and/or 1-specificity (true negative fraction). In one embodiment, the method is a Receiver Operating Characteristic (ROC), which provides several parameters to evaluate both the sensitivity and the specificity of the result of the equation generated. In one embodiment, the ROC area (area under the curve) is used to evaluate the equations. A ROC area greater than 0.5, 0.6, 0.7, 0.8, 0.9 is preferred. In some embodiments, the ROC is at least or about 0.91, 0.92, 0.93, 0.94, 0.95, 0.96, 0.97, 0.98, or 0.99. In some embodiments, the ROC is at least or about 0.9857. A perfect ROC area score of 1.0 is indicative of both 100% sensitivity and 100% specificity. The ROC curve can be calculated by various statistical tools, including, but not limited to, Statistical Analysis System (SAS®), or R.

In some embodiments, mathematical models are selected on the basis of the evaluation score. In some embodiments, where specificity is important, a sensitivity threshold can be set, and mathematical models ranked on the basis of the specificity are chosen. For example, mathematical models with a cutoff for specificity of greater than 0.95, 0.9, 0.85, 0.8, 0.7, 0.65, 0.6, 0.55 0.5 or 0.45 can be chosen. Similarly, the specificity threshold can be set, and mathematical models ranked on the basis of sensitivity (e.g., greater than 0.95, 0.9, 0.85, 0.8, 0.7, 0.65, 0.6, 0.55 0.5 or 0.45) can be chosen. Thus, in some embodiments, only the top ten ranking mathematical models, the top twenty ranking mathematical models, or the top one hundred ranking mathematical models are selected.

A person skilled in the art will appreciate that the sensitivity and the specificity depend on the selected reference threshold (or the cut-off point). The more stringent the reference threshold, the lower the sensitivity and the higher the specificity. The reference threshold can be optimized for the sensitivity, the specificity, or the percentage of correct predictions. Thus, a reference threshold can be set based on the desired sensitivity and/or the desired specificity.

In some embodiments, accuracy, specificity, sensitivity, precision (positive predictive value), negative predictive value and F1-Score can be calculated. In some embodiments, the mathematical model has an outstanding performance with a value for accuracy, specificity, sensitivity, precision, negative predictive value, and/or F1-score that is about or at least 0.99, 0.98, 0.97, 0.96, 0.95, 0.94, 0.93, 0.92, 0.91, 0.9, 0.85, or 0.8.

In some embodiments, the methods as described herein can increase accuracy, specificity, sensitivity, precision (positive predictive value), negative predictive value and/or F1-Score by at least 10%, 20%, 30%, 40%, 50%, 60%, 70%, 80%, or 90%, as compared to a method that is commonly used in the field.

### Sample Preparation

Provided herein are methods and compositions for analyzing nucleic acids. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, cell or tissue origins, tumor origins, cancer origins, sample origins, subject origins, fetal origins, maternal origins), or combinations thereof.

Nucleic acid or a nucleic acid mixture described herein can be isolated from a sample obtained from a subject. A subject can be any living or non-living organism, including but not limited to a human, a non-human animal, a mammal, a plant, a bacterium, a fungus or a virus. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject can be a male or female.

Nucleic acid can be isolated from any type of suitable biological specimen or sample (e.g., a test sample). A sample or test sample can be any specimen that is isolated or obtained from a subject (e.g., a human subject). Non-limiting examples of specimens include fluid or tissue from a subject, including, without limitation, blood, serum, umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample, celocentesis sample, fetal cellular remnants, urine, feces, sputum, saliva, nasal mucous, prostate fluid, lavage, semen, lymphatic fluid, bile, tears, sweat, breast milk, breast fluid, embryonic cells and fetal cells (e.g. placental cells).

In some embodiments, a biological sample can be blood, plasma or serum. As used herein, the term “blood” encompasses whole blood or any fractions of blood, such as serum and plasma. Blood or fractions thereof can comprise cell-free or intracellular nucleic acids. Blood can comprise buffy coats. Buffy coats are sometimes isolated by utilizing a ficoll gradient. Buffy coats can comprise white blood cells (e.g., leukocytes, T-cells, B-cells, platelets). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation. A fluid or tissue sample from which nucleic acid is extracted can be acellular (e.g., cell-free). In some embodiments, a fluid or tissue sample can contain cellular elements or cellular remnants. In some embodiments, cancer cells or tumor cells can be included in the sample.

A sample often is heterogeneous. In many cases, more than one type of nucleic acid species is present in the sample. For example, heterogeneous nucleic acid can include, but is not limited to, cancer and non-cancer nucleic acid, pathogen and host nucleic acid, and/or mutated and wild-type nucleic acid. A sample may be heterogeneous because more than one cell type is present, such as a cancer and non-cancer cell, or a pathogenic and host cell.

In some embodiments, the sample comprise cell free DNA (cfDNA) or circulating tumor DNA (ctDNA). As used herein, the term “cell-free DNA” or “cfDNA” refers to DNA that is freely circulating in the bloodstream. These cfDNA can be isolated from a source having substantially no cells. In some embodiments, these extracellular nucleic acids can be present in and obtained from blood. Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species. For example, blood serum or plasma from a person having cancer can include nucleic acid from cancer cells and nucleic acid from non-cancer cells. As used herein, the term “circulating tumor DNA” or “ctDNA” refers to tumor-derived fragmented DNA in the bloodstream that is not associated with cells. ctDNA usually originates directly from the tumor or from circulating tumor cells (CTCs). The circulating tumor cells are viable, intact tumor cells that shed from primary tumors and enter the bloodstream or lymphatic system. The ctDNA can be released from tumor cells by apoptosis and necrosis (e.g., from dying cells), or active release from viable tumor cells (e.g., secretion). Studies show that the size of fragmented ctDNA is predominantly 166 bp long, which corresponds to the length of DNA wrapped around a nucleosome plus a linker. Fragmentation of this length might be indicative of apoptotic DNA fragmentation, suggesting that apoptosis may be the primary method of ctDNA release. Thus, in some embodiments, the length of ctDNA or cfDNA can be at least or about 70, 80, 90, 100, 110, 120, 130, 140, 150, 160, 170, 180, 190, or 200 bp. In some embodiments, the length of ctDNA or cfDNA can be less than about 70, 80, 90, 100, 110, 120, 130, 140, 150, 160, 170, 180, 190, or 200 bp. In some embodiments, the cell-free nucleic acid is of a length of about 500, 250, or 200 base pairs or less.

The present disclosure provides methods of separating, enriching and analyzing cell free DNA or circulating tumor DNA found in blood as a non-invasive means to detect the presence and/or to monitor the progress of a cancer. Thus, the first steps of practicing the methods described herein are to obtain a blood sample from a subject and extract DNA from the subject.

A blood sample can be obtained from a subject (e.g., a subject who is suspected to have cancer). The procedure can be performed in hospitals or clinics. An appropriate amount of peripheral blood, e.g., typically between 1 and 50 ml (e.g., between 1 and 10 ml), can be collected. Blood samples can be collected, stored or transported in a manner known to the person of ordinary skill in the art to minimize degradation or the quality of nucleic acid present in the sample. In some embodiments, the blood can be placed in a tube containing EDTA to prevent blood clotting, and plasma can then be obtained from whole blood through centrifugation. Serum can be obtained with or without centrifugation-following blood clotting. If centrifugation is used then it is typically, though not exclusively, conducted at an appropriate speed, e.g., 1,500-3,000×g. Plasma or serum can be subjected to additional centrifugation steps before being transferred to a fresh tube for DNA extraction.

In addition to the acellular portion of the whole blood, DNA can also be recovered from the cellular fraction, enriched in the buffy coat portion, which can be obtained following centrifugation of a whole blood sample.

There are numerous known methods for extracting DNA from a biological sample including blood. The general methods of DNA preparation (e.g., described by Sambrook and Russell, Molecular Cloning: A Laboratory Manual 3d ed., 2001) can be followed; various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), may also be used to obtain DNA from a blood sample.

cfDNA purification is prone to contamination due to ruptured blood cells during the purification process. Because of this, different purification methods can lead to significantly different cfDNA extraction yields. In some embodiments, purification methods involve collection of blood via venipuncture, centrifugation to pellet the cells, and extraction of cfDNA from the plasma. In some embodiments, after extraction, cell-free DNA can be about or at least 50% of the overall nucleic acid (e.g., about or at least 50%, 60%, 70%, 80%, 90%, 91%, 92%, 93%, 94%, 95%, 96%, 97%, 98%, or 99% of the total nucleic acid is cell-free DNA).

The nucleic acid that can be analyzed by the methods described herein include, but are not limited to, DNA (e.g., complementary DNA (cDNA), genomic DNA (gDNA), cfDNA, or ctDNA), ribonucleic acid (RNA) (e.g., message RNA (mRNA), short inhibitory RNA (siRNA), ribosomal RNA (rRNA), transfer RNA (tRNA), or microRNA), and/or DNA or RNA analogs (e.g., containing base analogs, sugar analogs and/or a non-native backbone and the like), RNA/DNA hybrids and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, or double-stranded). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures.

Nucleic acid provided for processes described herein can contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

In some embodiments, the nucleic acid can be extracted, isolated, purified, partially purified or amplified from the samples before sequencing. In some embodiments, nucleic acid can be processed by subjecting nucleic acid to a method that generates nucleic acid fragments. Fragments can be generated by a suitable method known in the art, and the average, mean or nominal length of nucleic acid fragments can be controlled by selecting an appropriate fragment-generating procedure. In certain embodiments, nucleic acid of a relatively shorter length can be utilized to analyze sequences that contain little sequence variation and/or contain relatively large amounts of known nucleotide sequence information. In some embodiments, nucleic acid of a relatively longer length can be utilized to analyze sequences that contain greater sequence variation and/or contain relatively small amounts of nucleotide sequence information.

### Sequencing

Nucleic acids (e.g., nucleic acid fragments, sample nucleic acid, cell-free nucleic acid, circulating tumor nucleic acids) are sequenced before the analysis.

As used herein, “reads” or “sequence reads” are short nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads).

Sequence reads obtained from cell-free DNA can be reads from a mixture of nucleic acids derived from normal cells or tumor cells. A mixture of relatively short reads can be transformed by processes described herein into a representation of a genomic nucleic acid present in a subject. In certain embodiments, “obtaining” nucleic acid sequence reads of a sample can involve directly sequencing nucleic acid to obtain the sequence information.

Sequence reads can be mapped and the number of reads or sequence tags mapping to a specified nucleic acid region (e.g., a chromosome, a bin, a genomic section) are referred to as counts. In some embodiments, counts can be manipulated or transformed (e.g., normalized, combined, added, filtered, selected, averaged, derived as a mean, the like, or a combination thereof).

In some embodiments, a group of nucleic acid samples from one individual are sequenced. In certain embodiments, nucleic acid samples from two or more samples, wherein each sample is from one individual or two or more individuals, are pooled and the pool is sequenced together. In some embodiments, a nucleic acid sample from each biological sample often is identified by one or more unique identification tags.

The nucleic acids can also be sequenced with redundancy. A given region of the genome or a region of the cell-free DNA can be covered by two or more reads or overlapping reads (e.g., “fold” coverage greater than 1). Coverage (or depth) in DNA sequencing refers to the number of unique reads that include a given nucleotide in the reconstructed sequence. In some embodiments, a fraction of the genome is sequenced, which sometimes is expressed in the amount of the genome covered by the determined nucleotide sequences (e.g., “fold” coverage less than 1). Thus, in some embodiments, the fold is calculated based on the entire genome. When a genome is sequenced with about 1-fold coverage, roughly 100% of the nucleotide sequence of the genome is represented by reads. In some embodiments, cell free DNAs are sequenced and the fold is calculated based on the entire genome. Thus, it is easier to compare the amount of sequencing and the amount of sequencing reads that are generated for different projects.

The fold can also be calculated based on the length of the reconstructed sequence (e.g., cfDNA). When the cell free DNA is sequenced with about 1-fold coverage that is calculated based on the reconstructed sequence (e.g., panel sequencing), the number of nucleotides in all unique reads would be roughly the same as the entire nucleotide sequence of the cfDNA in the sample.

In some embodiments, the nucleic acid is sequenced with about 0.1-fold to about 100-fold coverage, about 0.2-fold to 20-fold coverage, or about 0.2-fold to about 1-fold coverage. In some embodiments, sequencing is performed by about or at least 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 200, 300, 400, 500, or 1000 fold coverage. In some embodiments, sequencing is performed by no more than 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 200, 300, 400, 500, or 1000 coverage. In some embodiments, sequencing is performed by no more than 15, 20, 30, 40, 50, 60, 70, 80, 90 or 100 fold coverage.

In some embodiments, the sequence coverage is performed by about or at least 0.2, 0.3, 0.4, 0.5, 0.6, 0.7, 0.8, 0.9, 1, 2, 3, 4, or 5 fold (e.g., as determined by the entire genome). In some embodiments, the sequence coverage is performed by no more than 0.2, 0.3, 0.4, 0.5, 0.6, 0.7, 0.8, 0.9, 1, 2, 3, 4, or 5 fold (e.g., as determined by the entire genome).

In some embodiments, the sequence coverage is performed by about or at least 100, 150, 200, 250, 300, 350, 400, 450, or 500 fold (e.g., as determined by reconstructed sequence). In some embodiments, the sequence coverage is performed by no more than 100, 150, 200, 250, 300, 350, 400, 450, or 500 fold (e.g., as determined by reconstructed sequence).

In some embodiments, a sequencing library can be prepared prior to or during a sequencing process. Methods for preparing the sequencing library are known in the art and commercially available platforms may be used for certain applications. Certain commercially available library platforms may be compatible with sequencing processes described herein. For example, one or more commercially available library platforms may be compatible with a sequencing by synthesis process. In certain embodiments, a ligation-based library preparation method is used (e.g., ILLUMINA TRUSEQ, Illumina, San Diego Calif.). Ligation-based library preparation methods typically use a methylated adaptor design which can incorporate an index sequence at the initial ligation step and often can be used to prepare samples for single-read sequencing, paired-end sequencing and multiplexed sequencing. In certain embodiments, a transposon-based library preparation method is used (e.g., EPICENTRE NEXTERA, Epicentre, Madison Wis.). Transposon-based methods typically use in vitro transposition to simultaneously fragment and tag DNA in a single-tube reaction (often allowing incorporation of platform-specific tags and optional barcodes), and prepare sequencer-ready libraries.

Any sequencing method suitable for conducting methods described herein can be used. In some embodiments, a high-throughput sequencing method is used. High-throughput sequencing methods generally involve clonally amplified DNA templates or single DNA molecules that are sequenced in a massively parallel fashion within a flow cell. Such sequencing methods also can provide digital quantitative information, where each sequence read is a countable “sequence tag” or “count” representing an individual clonal DNA template, a single DNA molecule, bin or chromosome.

Next generation sequencing techniques capable of sequencing DNA in a massively parallel fashion are collectively referred to herein as “massively parallel sequencing” (MPS). High-throughput sequencing technologies include, for example, sequencing-by-synthesis with reversible dye terminators, sequencing by oligonucleotide probe ligation, pyrosequencing and real time sequencing. Non-limiting examples of MPS include Massively Parallel Signature Sequencing (MPSS), Polony sequencing, Pyrosequencing, Illumina (Solexa) sequencing, SOLiD sequencing, Ion semiconductor sequencing, DNA nanoball sequencing, Helioscope single molecule sequencing, single molecule real time (SMRT) sequencing, nanopore sequencing, ION Torrent and RNA polymerase (RNAP) sequencing. Some of these sequencing methods are described e.g., in US20130288244A1, which is incorporated herein by reference in its entirety.

Systems utilized for high-throughput sequencing methods are commercially available and include, for example, the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used in high-throughput sequencing approaches.

The length of the sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, the sequence reads are of a mean, median or average length of about 15 bp to 900 bp long (e.g., about or at least 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp). In some embodiments, the sequence reads are of a mean, median or average length of about 1000 bp or more. In some embodiments, the sequence reads are of less than 60 bp, 65 bp, 70 bp, 75 bp, 80 bp, 85 bp, 90 bp, 95 bp, 100 bp, 110 bp, 120 bp, 130, 140 bp, 150 bp, 200 bp, 250 bp, 300 bp, 350 bp, 400 bp, 450 bp, or 500 bp are removed because of poor quality.

Mapping nucleotide sequence reads (i.e., sequence information from a fragment whose physical genomic position is unknown) can be performed in a number of ways, and often comprises alignment of the obtained sequence reads with a matching sequence in a reference genome (e.g., Li et al., “Mapping short DNA sequencing reads and calling variants using mapping quality score,” Genome Res., 2008 Aug. 19.) In such alignments, sequence reads generally are aligned to a reference sequence and those that align are designated as being “mapped” or a “sequence tag.” In certain embodiments, a mapped sequence read is referred to as a “hit” or a “count”.

As used herein, the terms “aligned”, “alignment”, or “aligning” refer to two or more nucleic acid sequences that can be identified as a match (e.g., 100% identity) or partial match. Alignments can be done manually or by a computer algorithm, examples including the Efficient Local Alignment of Nucleotide Data (ELAND) computer program distributed as part of the Illumina Genomics Analysis pipeline. The alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand. In certain embodiments, a nucleic acid sequence is aligned with the reverse complement of another nucleic acid sequence.

Various computational methods can be used to map each sequence read to a genomic region. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database. Search hits can then be used to sort the identified sequences into appropriate genomic sections, for example. Some of the methods of analyzing sequence reads are described e.g., US20130288244A1, which is incorporated herein by reference in its entirety.

### Detecting Cancer

The present disclosure provides methods of detecting and/or treating cancer.

In some embodiments, sequencing cell free DNA permits broader inquiries, allowing assessment of the mutation status of thousands/millions of positions. In some embodiments, detection of mutations at oncogenes or tumor suppressor genes indicate that the subject is likely to have cancer.

In some embodiments, mutations in the oncogenes can include one or more mutations at one or more oncogenes (e.g., TERT, ABL1 (ABL), ABL2 (ABLL, ARG), AKAP13 (HT31, LBC. BRX), ARAF1, ARHGEF5 (TIM), ATF1, AXL, BCL2, BRAF (BRAF1, RAFB1), BRCA1, BRCA2(FANCD1), BRIP1, CBL (CBL2), CSF1R (CSF-1, FMS, MCSF), DAPK1 (DAPK), DEK (D6S231E), DUSP6(MKP3,PYST1), EGF, EGFR (ERBB, ERBB1), ERBB3 (HER3), ERG, ETS1, ETS2, EWSR1 (EWS, ES, PNE,), FES (FPS), FGF4 (HSTF1, KFGF), FGFR1, FGFR10P (FOP), FLCN, FOS (c-fos), FRAP1, FUS (TLS), HRAS, GLI1, GLI2, GPC3, HER2 (ERBB2, TKR1, NEU), HGF (SF), IRF4 (LSIRF, MUM1), JUNB, KIT(SCFR), KRAS2 (RASK2), LCK, LCO, MAP3K8(TPL2, COT, EST), MCF2 (DBL), MDM2, MET(HGFR, RCCP2), MLH type genes, MMD, MOS (MSV), MRAS (RRAS3), MSH type genes, MYB (AMV), MYC, MYCL1 (LMYC), MYCN, NCOA4 (ELE1, ARA70, PTC3), NF1 type genes, NMYC, NRAS, NTRK1 (TRK, TRKA), NUP214 (CAN, D9S46E), OVC, TP53 (P53), PALB2, PAX3 (HUP2) STAT1, PDGFB (SIS), PIM genes, PML (MYL), PMS (PMSL) genes, PPM1D (WIP1), PTEN (MMAC1), PVT1, RAF1 (CRAF), RB1 (RB), RET, RRAS2 (TC21), ROS1 (ROS, MCF3), SMAD type genes, SMARCB1(SNF5, INI1), SMURF1, SRC (AVS), STAT1, STAT3, STAT5, TDGF1 (CRGF), TGFBR2, THRA (ERBA, EAR7 etc), TFG (TRKT3), TIF1 (TRIM24, TIF1A), TNC (TN, HXB), TRK, TUSC3, USP6 (TRE2), WNT1 (INT1), WT1, VHL). In some embodiments, mutations in the tumor suppressor genes include one or more mutations at one or more tumor suppressor genes (e.g., APC, BRCA1, BRCA2(FANCD1), CAPG, CDKN1A (CIP1, WAF1, p21), CDKN2A (CDKN2, MTS1(depreciated), TP16, p16(INK4)), CD99 (MIC2, MIC2X), FRAP1 (FRAP, MTOR, RAFT1), NF1, NF2, PI5, PDGFRL (PRLTS, PDGRL), PML (MYL), PPARG, PRKAR1A (TSE1), PRSS11 (HTRA, HTRA1)), PTEN (MMAC1), RRAS, RB1 (RB), SEMA3B, SMAD2 (MADH2, MADR2), SMAD3 (MADH3), SMAD4 (MADH4, DPC4), SMARCB1 (SNF5, INI1), ST3 (TSHL, CCTS), TET2, TOP1, TNC (TN, HXB), TP53 (P53), TP63 (TP73L), TP73, TSG11, TUSC2 (FUS1), TUSC3, VHL).

In some embodiments, the methods involve detection of specific mutations at oncogenes and/or tumor suppressor genes, e.g., detection of one or more mutations in EGFR, KRAS, TP53, IDH1, PIK3CA, BRAF, and/or NRAS. Some of these mutations are described e.g., in Mehrotra et al. “Detection of somatic mutations in cell-free DNA in plasma and correlation with overall survival in patients with solid tumors.” Oncotarget 9.12 (2018): 10259, which is incorporated herein by reference in its entirety.

In some embodiments, copy number variations and structural variants in the oncogenes and/or tumor suppressor genes indicate that the subject is likely to have cancer.

In some embodiments, mutation burden is used to detect cancer. As used herein, the term “mutation burden” refers to the level, e.g., number, of an alteration (e.g., one or more alterations, e.g., one or more somatic alterations) per a preselected unit (e.g., per megabase) in a predetermined set of genes (e.g., in the coding regions of the predetermined set of genes). Mutation load can be measured, e.g., on a whole genome or exome basis, on the basis of a subset of genome or exome, or on cfDNA.

In certain embodiments, the mutation load measured on the basis of a subset of genome or exome can be extrapolated to determine a whole genome or exome mutation load.

In some embodiments, the tumor mutation burden are limited to non-synonymous mutations. In some embodiments, the tumor mutation burden are limited to oncogenes and/or tumor suppressor genes.

In certain embodiments, the mutation load is measured in a sample, e.g., a tumor sample (e.g., a tumor sample or a sample derived from a tumor), from a subject, e.g., a subject described herein. In certain embodiments, the mutation load is expressed as a percentile, e.g., among the mutation loads in samples from a reference population. In certain embodiments, the reference population includes patients having the same type of cancer as the subject. In other embodiments, the reference population includes patients who are receiving, or have received, the same type of therapy, as the subject. In some embodiments, a subject is likely to have cancer if the mutation load is higher than a reference threshold. The subject is less likely to have cancer if the mutation load is lower than a reference threshold.

In some embodiments, the mutation burden can determine sensitivity to a therapeutic agent, e.g., a checkpoint inhibitor (e.g., anti-PD-1 antibody). In some embodiments, the therapy is an immunotherapy.

Some of these methods involving tumor mutation burden are described e.g., in Rizvi et al. “Mutational landscape determines sensitivity to PD-1 blockade in non-small cell lung cancer.” Science 348.6230 (2015): 124-128; Addeo et al., “Measuring tumor mutation burden in cell-free DNA: advantages and limits.” Translational Lung Cancer Research (2019), which are incorporated herein by reference in the entirety.

In some aspects, the methods described herein can also be used to detect recurrence. Thus, the methods described herein can be used to predict eventual recurrence, e.g., after surgery, chemotherapy, or some other curative treatments.

In some aspects, the methods described herein can also be used to evaluate treatment response and progression. Sequencing cell free DNA or circulating tumor DNA can be used to guide the choice of therapeutic agent and to monitor dynamic tumor responses throughout treatment. For example, the reemergence or significant increase in plasma tumor DNA during drug treatment, is strongly correlated with radiographic/clinical progression. Thus, in some embodiments, a decrease of plasma tumor DNA (while tumor or cancer symptoms persist) after the significant increase suggests the development of drug resistance, and the need of switching therapies. Some of these methods are described, e.g., in Ulrich et al, “Cell-free DNA in oncology: gearing up for clinic.” Annals of laboratory medicine 38.1 (2018): 1-8; Babayan et al., “Advances in liquid biopsy approaches for early detection and monitoring of cancer.” Genome medicine 10.1 (2018): 21, which are incorporated herein by reference in the entirety.

In some embodiments, certain medical procedures can be performed if a subject is identified as having an increased risk of having cancer. In some embodiments, these medical procedures can further confirm whether the subject has cancer. Some embodiments further include imaging procedures (e.g., CT scan, nuclear scan, ultrasound, MRI, PET scan, X-rays), biopsy (e.g., with a needle, with an endoscope, with surgery, excisional biopsy, incisional biopsy), or further lab tests (e.g., testing blood, urine, or other body fluids).

Some embodiments further include updating or recording the subject's risk of a cancer (e.g., a subject's increased risk of having cancer or tumor) in a clinical record or database. Some embodiments further include performing increased monitoring on a subject identified as having an increased risk of a cancer (e.g., increased periodicity of physical examination, and increased frequency of clinic visits). Some embodiments further include recording the need for increased monitoring in a clinical record or database for a subject identified as having an increased risk of having cancer. Some embodiments further include informing the subject to self-monitor for the symptoms of cancer. Some embodiments of the methods described herein include recommending a lifestyle change. Some of the lifestyle change include, but are not limited to, dietary change (e.g., eating more fruits and vegetables, eating less red meat, reduce alcohol consumption), taking vaccination (e.g., taking human papillomavirus vaccine, or hepatitis B vaccine), taking medications (e.g., nonsteroidal anti-inflammatory drug, COX-2 inhibitors, tamoxifen or raloxifene), lose weight, and/or do more exercise.

### Methods of Treatment

The present disclosure provides methods of treating a disease or a disorder as described herein. In some embodiments, the disease or the disorder is cancer. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject. In some embodiments, the compositions and methods disclosed herein can be used for treatment of patients at risk for a cancer.

The treatments can generally include e.g., surgery, chemotherapy, radiation therapy, hormonal therapy, targeted therapy, and/or a combination thereof. Which treatments are used depends on the type, location and grade of the cancer as well as the patient's health and preferences. In some embodiments, the therapy is chemotherapy or chemoradiation.

In one aspect, the disclosure features methods that include administering a therapeutically effective amount of a therapeutic agent to the subject in need thereof (e.g., a subject having, or identified or diagnosed as having, a cancer). In some embodiments, the subject has e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has triple-negative breast cancer (TNBC), gastric cancer, urothelial cancer, Merkel-cell carcinoma, or head and neck cancer.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent is to be administered, a severity of symptoms and a route of administration, and thus administration can be determined on an individual basis. An effective amount can be administered in one or more administrations. By way of example, an effective amount is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro.

In some embodiments, the methods described herein can be used to monitor the progression of the disease, determine the effectiveness of the treatment, and adjust treatment strategy. For example, cell free DNA can be collected from the subject to detect cancer and the information can also be used to select appropriate treatment for the subject. After the subject receives a treatment, cell free DNA can be collected from the subject. The analysis of these cfDNA can be used to monitor the progression of the disease, determine the effectiveness of the treatment, and/or adjust treatment strategy. In some embodiments, the results are then compared to the early results. In some embodiments, a dramatic increase of circulating tumor DNA indicates apoptosis at the tumor cells, which may suggest that the treatment is effective.

In some embodiments, the therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the therapeutic agent is an antibody or antigen-binding fragment thereof. In some embodiments, the therapeutic agent is an antibody that specifically binds to PD-1, CTLA-4, BTLA, PD-L1, CD27, CD28, CD40, CD47, CD137, CD154, TIGIT, TIM-3, GITR, or OX40.

In some embodiments, the therapeutic agent is an anti-PD-1 antibody, an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

In some embodiments, the therapeutic agent is an anti-CTLA4 antibody (e.g., ipilimumab), an anti-CD20 antibody (e.g., rituximab), an anti-EGFR antibody (e.g., cetuximab), an anti-CD319 antibody (e.g., elotuzumab), or an anti-PD1 antibody (e.g., nivolumab).

### Systems, Software, and Interfaces

The methods described herein (e.g., quantifying, mapping, normalizing, range setting, adjusting, categorizing, counting and/or determining sequence reads, and counts) often require a computer, processor, software, module or other apparatus. Methods described herein typically are computer-implemented methods, and one or more portions of a method sometimes are performed by one or more processors. Embodiments pertaining to methods described herein generally are applicable to the same or related processes implemented by instructions in systems, apparatus and computer program products described herein. In some embodiments, processes and methods described herein are performed by automated methods. In some embodiments, an automated method is embodied in software, modules, processors, peripherals and/or an apparatus comprising the like, that determine sequence reads, counts, mapping, mapped sequence tags, elevations, profiles, normalizations, comparisons, range setting, categorization, adjustments, plotting, outcomes, transformations and identifications. As used herein, software refers to computer readable program instructions that, when executed by a processor, perform computer operations, as described herein.

Sequence reads, counts, elevations, and profiles derived from a subject (e.g., a control subject, a patient or a subject is suspected to have tumor) can be analyzed and processed to determine the presence or absence of a genetic variation. Sequence reads and counts sometimes are referred to as “data” or “datasets”. In some embodiments, data or datasets can be characterized by one or more features or variables. In some embodiments, the sequencing apparatus is included as part of the system. In some embodiments, a system comprises a computing apparatus and a sequencing apparatus, where the sequencing apparatus is configured to receive physical nucleic acid and generate sequence reads, and the computing apparatus is configured to process the reads from the sequencing apparatus. The computing apparatus sometimes is configured to determine the presence or absence of a genetic variation (e.g., copy number variation, mutations) from the sequence reads.

Implementations of the subject matter and the functional operations described herein can be implemented in digital electronic circuitry, in tangibly-embodied computer software or firmware, in computer hardware, including the structures described herein and their structural equivalents, or in combinations of one or more of the structures. Implementations of the subject matter described herein can be implemented as one or more computer programs, i.e., one or more modules of computer program instructions encoded on a tangible program carrier for execution by, or to control the operation of, a processing device. Alternatively, or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

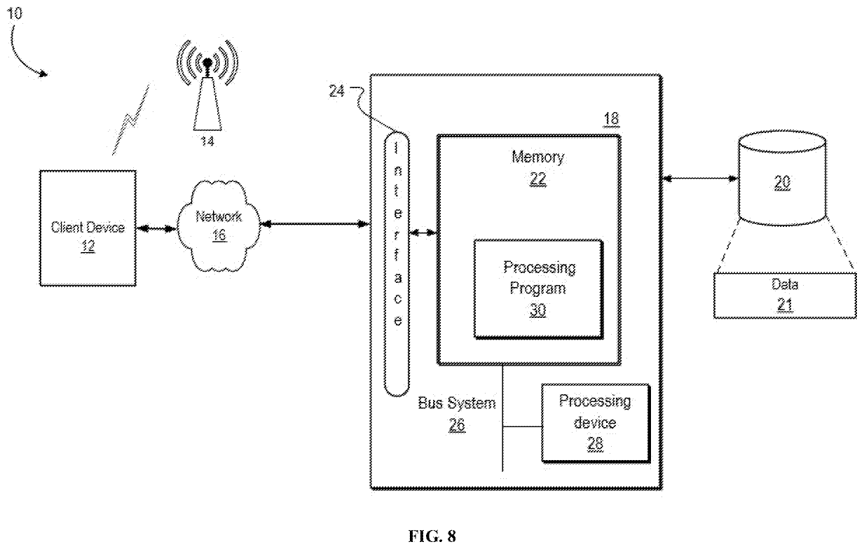

Referring to FIG. 8, system 10 processes data via binding data to parameters and applying a sequencing noise processor to the input data, and outputs information (e.g., quality score, Information Score) indicative of sequencing noise. System 10 includes client device 12, data processing system 18, data repository 20, network 16, and wireless device 14. The sequencing noise processor processes the input data based on the methods described herein. In some embodiments, the sequencing noise processor generates a quality score (e.g., information score) based on the methods described herein.

Data processing system 18 retrieves, from data repository 20, data 21 representing one or more values for the sequencing noise processor parameter, including e.g., the nucleotide frequency in control samples, the nucleotide frequency in tumor samples, and the background frequency in the whole human genome, etc. Data processing system 18 inputs the retrieved data into a sequencing noise processor, e.g., into data processing program 30. In this embodiment, data processing program 30 is programmed to detect sequencing noise. In some embodiments, the sequencing noise is detected by calculating information score, Log Odds Product Score, and Log Odds Sum score as described herein.

In some embodiments, data processing system 18 binds to parameter one or more values representing information associated with the variant (e.g., allele frequency at a position of interest). Data processing system 18 binds values of the data to the parameter by modifying a database record such that a value of the parameter is set to be the value of data 21 (or a portion thereof). Data 21 includes a plurality of data records that each have one or more values for the parameter. In some embodiments, data processing system 18 applies data processing program 30 to each of the records by applying data processing program 30 to the bound values for the parameter. Based on application of data processing program 30 to the bound values (e.g., as specified in data 21 or in records in data 21), data processing system 18 determines a score indicating whether the variant is likely to be a true mutation or sequencing noise. In some embodiments, data processing system 18 outputs, e.g., to client device 12 via network 16 and/or wireless device 14, data indicative of the determined quality score, or data indicating whether a variant is a true mutation or sequencing noise.

In some embodiments, based on the data indicating whether a variant is a true mutation or sequencing noise, data processing system 18 can be configured to determine whether a subject has cancer or is at risk of having cancer. If the data processing system 18 determines that the subject has cancer or is at risk of having cancer, data processing system 18 can further update a clinical record in the data 21, indicating the subject has cancer or is at risk of having cancer. In some embodiments, the record includes the need of performing increased monitoring (e.g., increased periodicity of physical examination, and increased frequency of clinic visits), the need for further procedures (e.g., diagnostics, lab tests, or treatment procedures), and recommendation for a lifestyle change.

Data processing system 18 generates data for a graphical user interface that, when rendered on a display device of client device 12, display a visual representation of the output. In some embodiments, the values for these parameters can be stored in data repository 20 or memory 22.

Client device 12 can be any sort of computing device capable of taking input from a user and communicating over network 16 with data processing system 18 and/or with other client devices. Client device 12 can be a mobile device, a desktop computer, a laptop computer, a cell phone, a personal digital assistant (PDA), a server, an embedded computing system, and so forth.

Data processing system 18 can be any of a variety of computing devices capable of receiving data and running one or more services. In some embodiments, data processing system 18 can include a server, a distributed computing system, a desktop computer, a laptop computer, a cell phone, and the like. Data processing system 18 can be a single server or a group of servers that are at a same position or at different positions (i.e., locations). Data processing system 18 and client device 12 can run programs having a client-server relationship to each other. Although distinct modules are shown in the figure, in some embodiments, client and server programs can run on the same device.

Data processing system 18 can receive data from wireless device 14 and/or client device 12 through input/output (I/O) interface 24 and data repository 20. Data repository 20 can store a variety of data values for data processing program 30. The sequencing noise processing program (which may also be referred to as a program, software, a software application, a script, or code) can be written in any form of programming language, including compiled or interpreted languages, or declarative or procedural languages, and it can be deployed in any form, including as a stand-alone program or as a module, component, subroutine, or other unit suitable for use in a computing environment. The data processing program may, but need not, correspond to a file in a file system. The program can be stored in a portion of a file that holds other programs or information (e.g., one or more scripts stored in a markup language document), in a single file dedicated to the program in question, or in multiple coordinated files (e.g., files that store one or more modules, sub programs, or portions of code). The data processing program can be deployed to be executed on one computer or on multiple computers that are located at one site or distributed across multiple sites and interconnected by a communication network.

In some embodiments, data repository 20 stores data 21 indicative of sequencing reads of samples from control subjects and sequencing reads of samples from tumor patients or patients who are suspected to have tumor. In another embodiment, data repository 20 stores parameters of the sequencing noise processor. Interface 24 can be a type of interface capable of receiving data over a network, including, e.g., an Ethernet interface, a wireless networking interface, a fiber-optic networking interface, a modem, and so forth. Data processing system 18 also includes a processing device 28. As used herein, a “processing device” encompasses all kinds of apparatuses, devices, and machines for processing information, such as a programmable processor, a computer, or multiple processors or computers. The apparatus can include special purpose logic circuitry, e.g., an FPGA (field programmable gate array) or an ASIC (application specific integrated circuit) or RISC (reduced instruction set circuit). The apparatus can also include, in addition to hardware, code that creates an execution environment for the computer program in question, e.g., code that constitutes processor firmware, a protocol stack, an information base management system, an operating system, or a combination of one or more of them.

Data processing system 18 also includes a memory 22 and a bus system 26, including, for example, a data bus and a motherboard, which can be used to establish and to control data communication between the components of data processing system 18. Processing device 28 can include one or more microprocessors. Generally, processing device 28 can include an appropriate processor and/or logic that is capable of receiving and storing data, and of communicating over a network. Memory 22 can include a hard drive and a random access memory storage device, including, e.g., a dynamic random access memory, or other types of non-transitory, machine-readable storage devices. Memory 22 stores data processing program 30 that is executable by processing device 28. These computer programs may include a data engine for implementing the operations and/or the techniques described herein. The data engine can be implemented in software running on a computer device, hardware or a combination of software and hardware.

Various methods and formulae can be implemented, in the form of computer program instructions, and executed by a processing device. Suitable programming languages for expressing the program instructions include, but are not limited to, C, C++, an embodiment of FORTRAN such as FORTRAN77 or FORTRAN90, Java, Visual Basic, Perl, Tcl/Tk, JavaScript, ADA, and statistical analysis software, such as SAS, R, MATLAB, SPSS, and Stata etc. Various aspects of the methods may be written in different computing languages from one another, and the various aspects are caused to communicate with one another by appropriate system-level-tools available on a given system.

The processes and logic flows described in this disclosure can be performed by one or more programmable computers executing one or more computer programs to perform functions by operating on input information and generating output. The processes and logic flows can also be performed by, and apparatus can also be implemented as, special purpose logic circuitry, e.g., an FPGA (field programmable gate array) or an ASIC (application specific integrated circuit) or RISC.

Computers suitable for the execution of a computer program include, by way of example, general or special purpose microprocessors, or both, or any other kind of central processing unit. Generally, a central processing unit will receive instructions and information from a read only memory or a random access memory or both. The essential elements of a computer are a central processing unit for performing or executing instructions and one or more memory devices for storing instructions and information. Generally, a computer will also include, or be operatively coupled to receive information from or transfer information to, or both, one or more mass storage devices for storing information, e.g., magnetic, magneto optical disks, or optical disks. However, a computer need not have such devices. Moreover, a computer can be embedded in another device, e.g., a mobile telephone, a smartphone or a tablet, a touchscreen device or surface, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

Implementations of the subject matter described herein can be implemented in a computing system that includes a back end component, e.g., as an information server, or that includes a middleware component, e.g., an application server, or that includes a front end component, e.g., a client computer having a graphical user interface or a Web browser through which a user can interact with an implementation of the subject matter, or any combination of one or more such back end, middleware, or front end components. The components of the system can be interconnected by any form or medium of digital information communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, the server can be in the cloud via cloud computing services.

While this disclosure includes many specific implementation details, these should not be construed as limitations on the scope of any of what may be claimed, but rather as descriptions of features that may be specific to particular implementations. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are described in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. In one embodiment, the processes depicted in the accompanying figures do not necessarily require the particular order shown, or sequential order, to achieve desirable results. In some implementations, multitasking and parallel processing may be advantageous.

### Kits

The present disclosure also provides kits for collecting, transporting, and/or analyzing samples. Such a kit can include materials and reagents required for obtaining an appropriate sample from a subject, or for measuring the levels of particular biomarkers. In some embodiments, the kits include those materials and reagents that would be required for obtaining and storing a sample from a subject. The sample is then shipped to a service center for further processing (e.g., sequencing and/or data analysis).

The kits may further include instructions for collect the samples, performing the assay and methods for interpreting and analyzing the data resulting from the performance of the assay.

### EXAMPLES

The invention is further described in the following examples, which do not limit the scope of the invention described in the claims.

### Example 1: Data Preparation

DNA in Tumor samples were sequenced by Illumina platform (e.g. X-10, NovaSeq). The qualities of raw output reads were checked by FastQC. The raw data would be trimmed by fastp to remove low-quality reads (any read having more than 40% of base quality less than 20 and any read shorter than 70 bp after all default trimming). Remaining data were checked by FastQC again to confirm that they still meet above criteria. Data passing QC after trimming were aligned by BWA (0.7.17-r1194-dirty). The output were converted by Samtools into BAM and PILEUP files. Finally, the score on each base in hg19 genome assembly was generated by in-house C++ implementation.

### I. Simulated Dataset

This dataset is generated by SeqMaker in OpenGene toolbox (Chen et al. “SeqMaker: A next generation sequencing simulator with variations, sequencing errors and amplification bias integrated.” 2016 IEEE International Conference on Bioinformatics and Biomedicine (BIBM). IEEE, 2016). The parameters were set up as the following:
(1) SeqMaker simulated NextGen sequencing data with 1000× depth on 93 genes.
(2) In every genes, only one true mutation was assigned. Its type and position was randomly determined, carrying an allele frequency ranging between 0.001 and 0.1.
   Due to randomness in data simulation, true mutations on 20 genes had no supporting reads at all. These 20 genes were not included in the following analyses.

### II. ROC Analysis

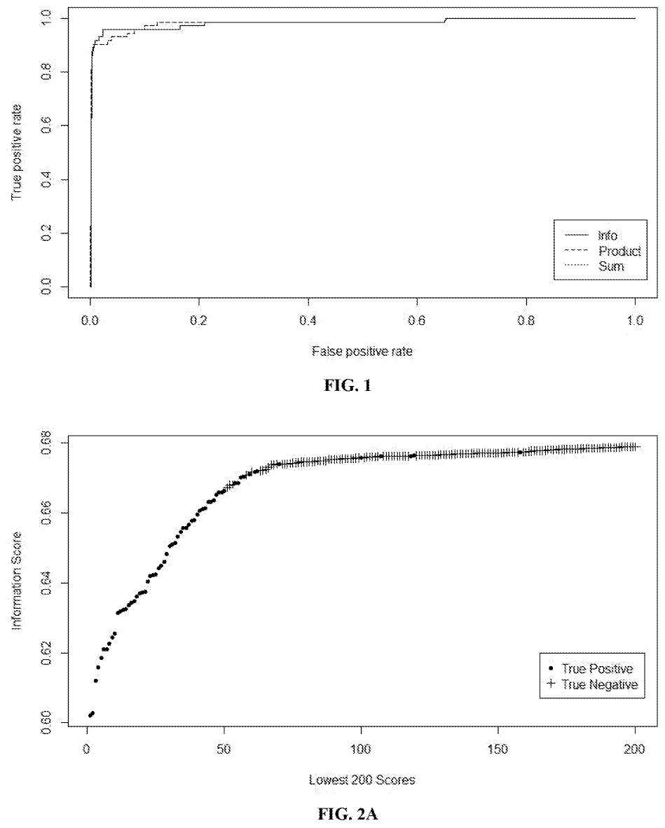

Information score, Log Odds Product Score, and Log Odds Sum Score were calculated for the remaining 73 genes based on simulated sequencing data. Only if the score of the true mutation topped those of all positions in the gene, it would be considered as a true positive. The ROC plot of these three scores was shown in FIG. 1. FIG. 1 showed that information score performed best in mutation detection on simulated ctDNA sequencing data.

### Example 2: Mutation Calling in Experiments

In the real data, the mutations need to be selected from all positions of all genes because it is unknown how many true mutations there are in one gene. Therefore, all positions of these 73 genes were sorted by their scores.

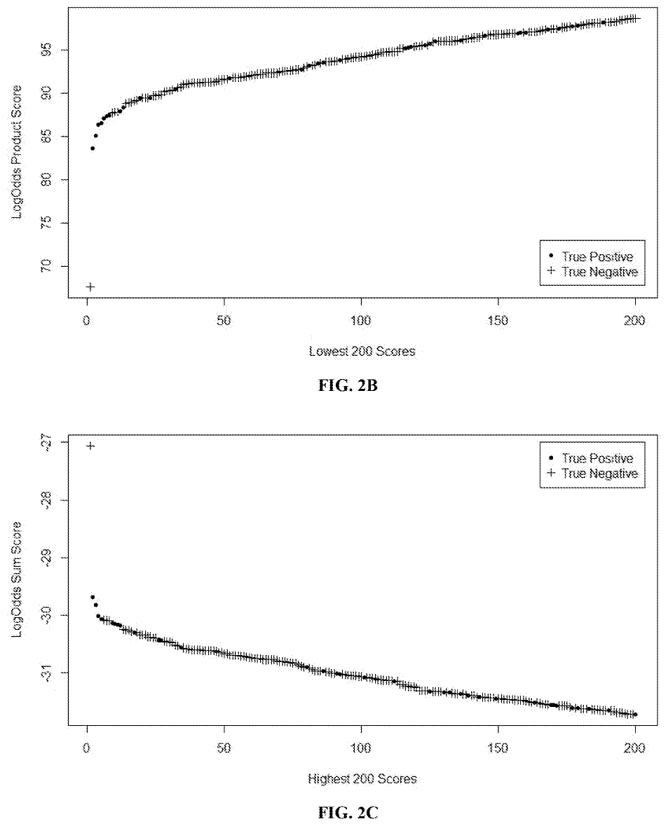

FIG. 2A shows the information score for the 200 mutation calls. The true positives were enriched among the mutations that had the lowest information scores.

FIG. 2B shows the Log Odds Product score for the 200 mutation calls with the lowest Log Odds Product score. As shown in FIG. 2B, the true positives were randomly distributed among these mutations.

FIG. 2C shows the Log Odds Sum score for the 200 mutation calls with the highest scores (lowest absolute value). A higher score indicates that the mutation is more likely to be a true positive. As shown in FIG. 2C, the true positives were randomly distributed among these mutations.

True positives and false positives were indicated in these figures.

The results in FIGS. 2A-2C show that information score performed the best to identify the true positives.

The results were also compared to TNER, a program that is commonly used to reduce background error for mutation detection in circulating tumor DNA (Deng et al. “TNER: a novel background error suppression method for mutation detection in circulating tumor DNA.” BMC bioinformatics 19.1 (2018): 387). The information score as described herein outperformed TNER. TNER recognized 51 true positives out of its 86 outputs. In contrast, information score identified 53 true positives out of the top 86 mutations.

### Example 3: Correlation with the Target Allele Frequency

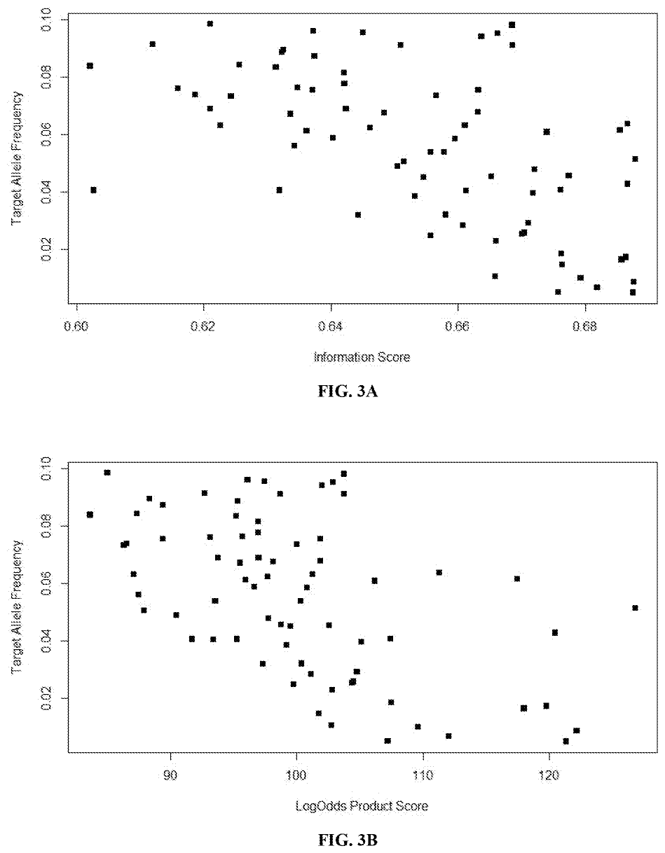

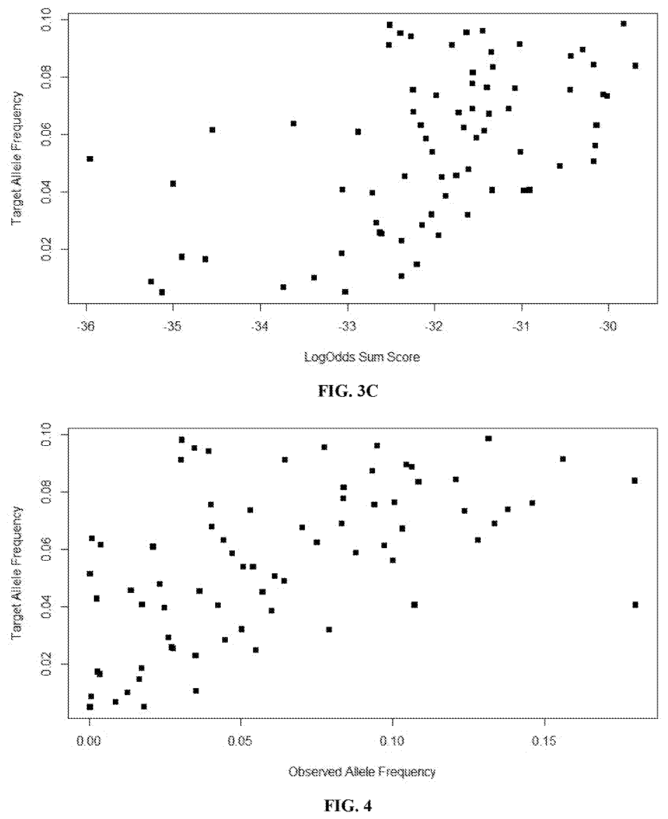

A score for mutation detection should capture the information of the target allele frequency as much as possible since the target allele frequency is an important criterion to detect a true mutation. FIGS. 3A-3C show how much information from the target allele frequency (i.e. correlation coefficient between the target allele frequencies and scores) can be obtained by these three different scores.

FIG. 3A shows the relationship between target allele frequency and information scores. The correlation coefficient is −0.572362.

FIG. 3B shows the relationship between target allele frequency and Log Odds Product Scores. The correlation coefficient is −0.5340896.

FIG. 3C shows the relationship between target allele frequency and Log Odds Sum Score. The correlation coefficient is 0.528966.

Information score again had the highest correlation with the target allele frequency. Thus, it is the the best estimator of the true mutation among the three scores. However, information score can achieve only 0.57 correlation coefficient (c.c.) with the target allele frequency, which is not surprising since the correlation coefficient between the observed allele frequency and the target allele frequency was 0.55 (FIG. 4). FIG. 4 shows the relationship between the observed allele frequency and the target allele frequency. The correlation coefficient is 0.554857. Information score achieved higher correlation coefficient than the observed allele frequency because it uses some information from the background to cancel out some noise.

### Example 3: Correlation with the Observed Allele Frequency

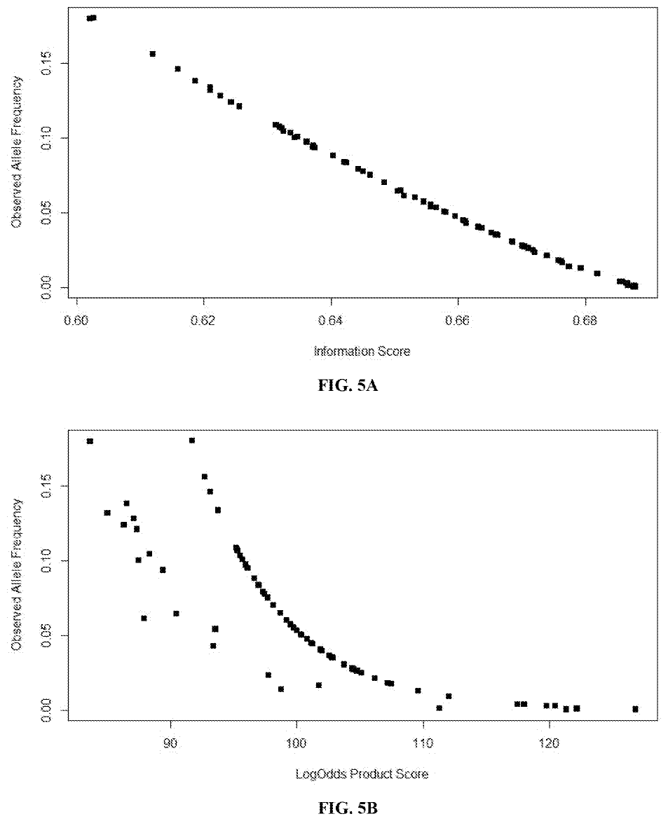

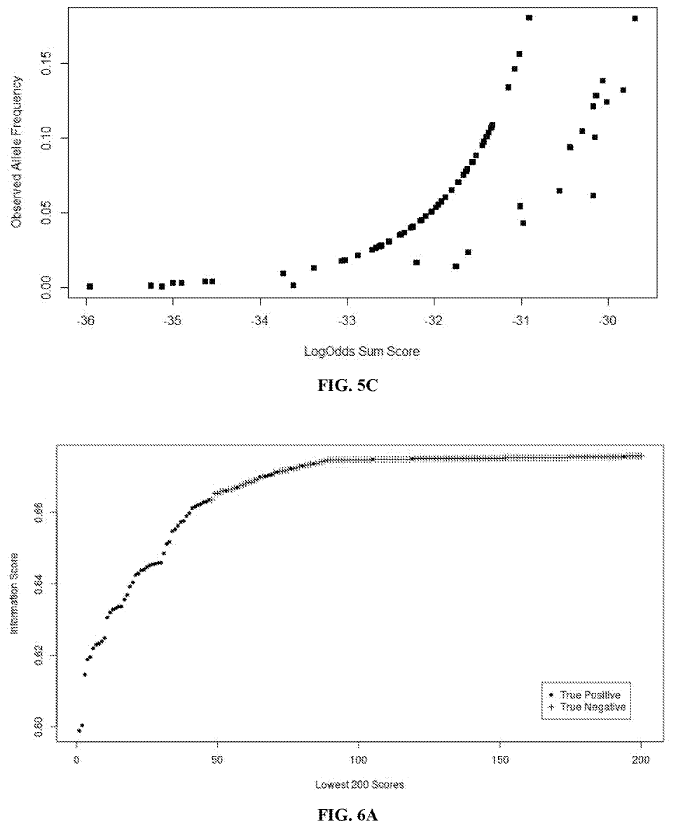

All of the three scores had high correlations with the observed allele frequencies, indicating their capabilities to capture the mutation information from sequencing reads (FIGS. 5A-5C). Among them, information score still outperformed the other two score.

FIG. 5A shows the relationship between information score and the observed allele frequency. The correlation coefficient is −0.995983.

FIG. 5B shows the relationship between Log Odds Product Score and the observed allele frequency. The correlation coefficient is −0.8240068.

FIG. 5C shows the relationship between Log odds Sum Score and the observed allele frequency. The correlation coefficient is 0.8092415.

Thus, information score had the highest correlation efficient (absolute value) with the observed allele frequency.

### Example 4: Performance Under Low Depth Sequencing

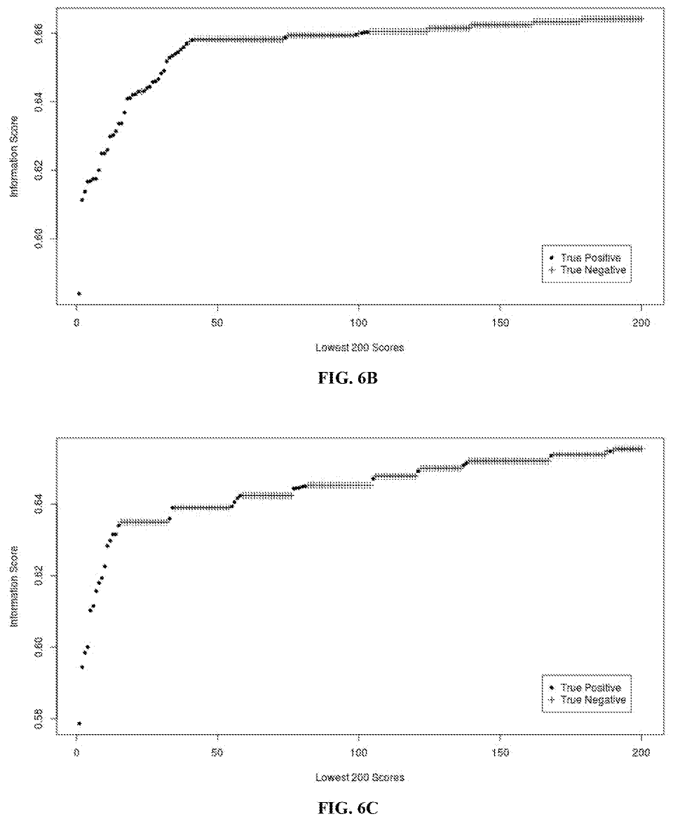

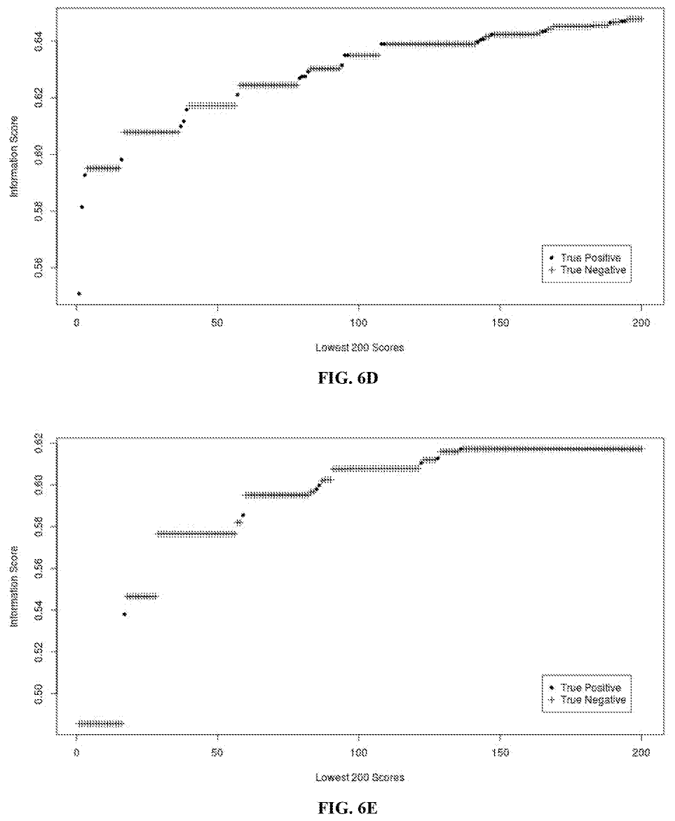

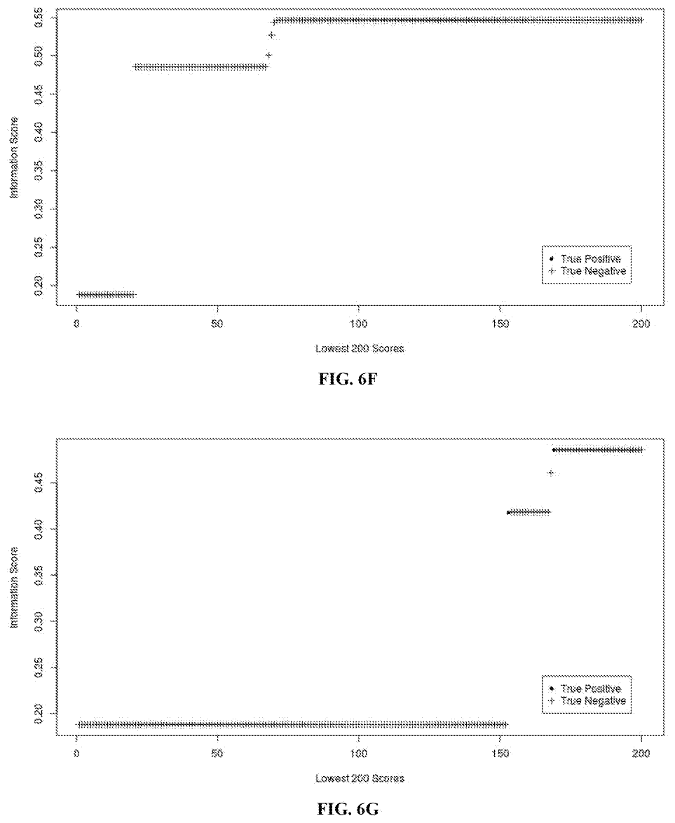

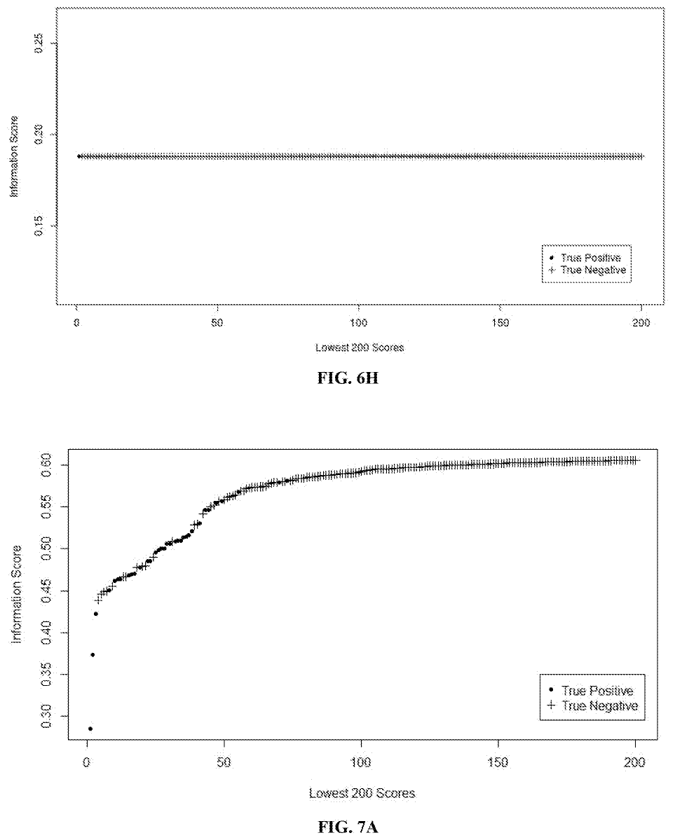

The results from the early examples show that information scores was the best estimator of the target allele frequency and the best criterion to call ctDNA mutations under the high depth (1000×) of simulated sequencing data. Experiments were also performed to test information score's performance for low depth sequencing data. The sequencing depth was decreased gradually. The results are shown FIGS. 6A-6H and the true positives were marked among the mutations with top scores. The results are summarized in the table below.

TABLE 4

| | True | Percentages of | | Percentages of true |
|---|---|---|---|---|
| Sequencing | positives in | true positives in | Total true | positives captured |
| coverage | top 200 | top 200 | positives | by top 200 |
| 500X | 50 | 25.0% | 72 | 69.4% |
| 200X | 40 | 20.0% | 68 | 58.8% |
| 100X | 30 | 15.0% | 65 | 46.2% |
| 50X | 23 | 11.5% | 54 | 42.6% |
| 20X | 7 | 3.5% | 40 | 17.5% |
| 10X | 0 | 0.0% | 30 | 0.0% |
| 5X | 2 | 1.0% | 11 | 18.2% |
| 2X | 1 | 0.5% | 5 | 20.0% |

FIGS. 6A-6H show that performance of information score decreased when the sequencing depth decreased. This suggests that higher sequencing depth would bring better performance generally.

### Example 5: Validation in Real Sequencing Data

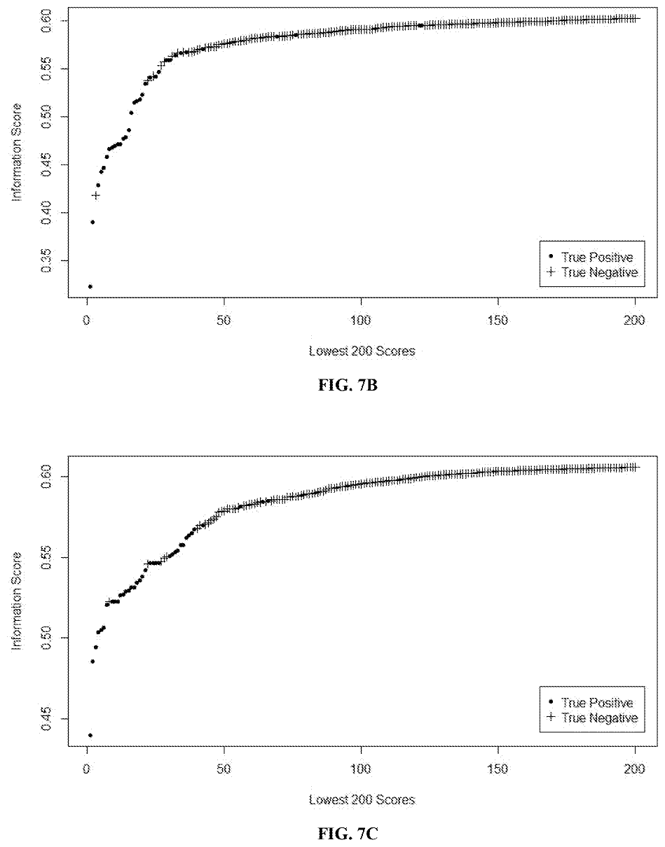

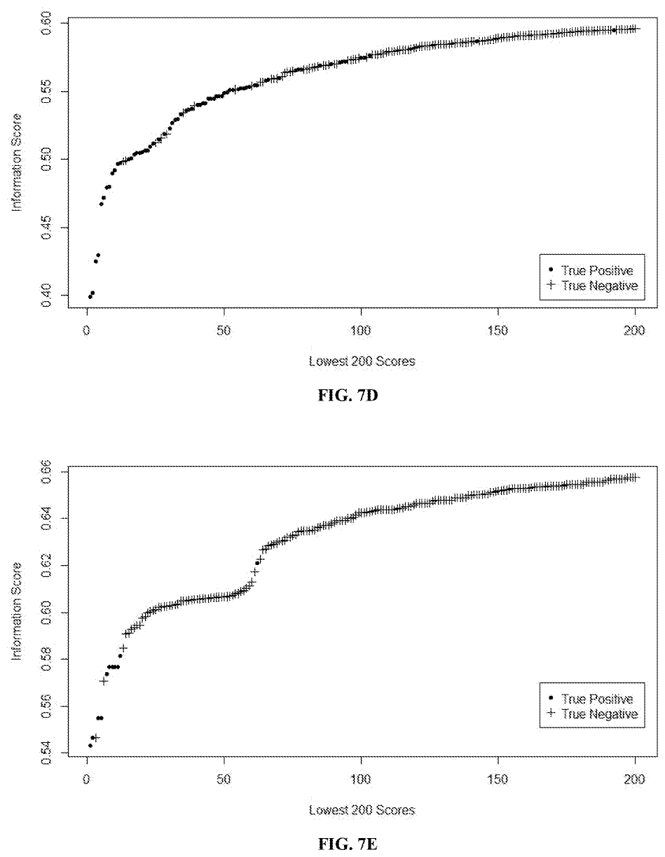

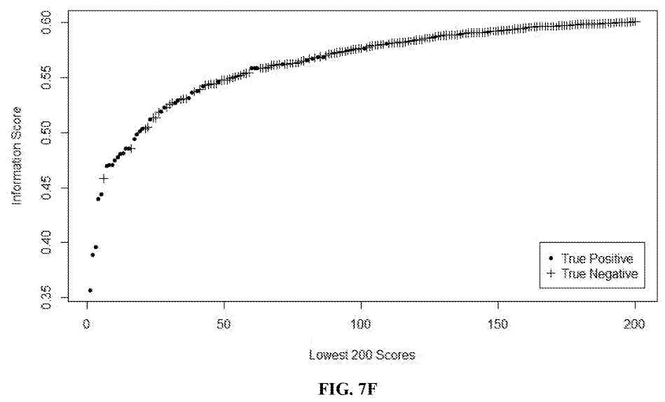

Performance of information score was further validated in real sequencing data provided by Asian Cancer Research Group (ACRG) project. Data from ACRG Subject ID 200, 11, 22, 26, 68 and 82 were selected for this validation test because these cases also provide some experiment-validated somatic variants as true positives. Information score on every validated somatic variant and their upstream and downstream 1000 bases was sorted in each ACRG case (FIGS. 7A-7F).

TABLE 5

| | | | | | Rank of the |
|---|---|---|---|---|---|
| | | | | | last true |
| | | | | Percentages of | positive |
| | | True positives | | true positives | among the |
| Subject | | in top 200 | Total true | captured by top | top 200 |
| ID | Depth | mutations | positives | 200 mutations | mutations |
| 200 | >20 | 33 | 33 | 100.00% | 62 |
| 11 | >20 | 26 | 27 | 96.30% | 106 |
| 22 | >20 | 37 | 37 | 100.00% | 63 |
| 26 | >20 | 69 | 70 | 98.57% | 192 |
| 68 | >20 | 10 | 10 | 100.00% | 61 |
| 82 | >20 | 37 | 37 | 100.00% | 108 |

The results confirmed the enrichment of true positives in top scores and proved that information score was a promising method to detect true somatic variants in real sequencing data.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for cancelling noise in sequencing results, the method comprising:
(a) determining frequencies for each base type in control samples and determining frequencies for each base type in a sample collected from a subject having a tumor or suspected to have a tumor at a position of interest in the genome;
(b) determining a divergence score for the position of interest by calculating mutual entropy between the distribution of base type frequencies in control samples and the distribution of base type frequencies in the sample collected from the subject having a tumor or suspected to have a tumor;
(c) determining a significance score by determining that probability that the distribution of base type frequencies in control samples and the distribution of base type frequencies in the sample collected from the subject having a tumor or suspected to have a tumor represent the same distribution;
(d) calculating an information score based on the divergence score and the significance score, wherein a higher information score indicates that the sequencing results at the position of interest is more likely to be noise.

2. The method of claim 1, wherein the sample is derived from whole blood, plasma and tissues, or saliva.

3. The method of claim 1, wherein the sample is circulating cell-free nucleic acids.

4. The method of claim 1, wherein the divergence score is calculated by the formula:
$${D}_{i} = \frac{1}{2} [ {\sum }_{j = 1}^{4} {}_{j}^{i}{Q}{}_{T}^{} {log}_{2} \frac{{}_{j}^{i}{Q}{}_{r}^{}}{{}_{j}^{i}{Q}{}_{v}^{}} + {\sum }_{j = 1}^{4} {}_{j}^{i}{Q}{}_{N}^{} {log}_{2} \frac{{}_{j}^{i}{Q}{}_{N}^{}}{{}_{j}^{i}{Q}{}_{v}^{}} ]$$
wherein ⁱⱼQ_{N} is the frequency for a base type j at position of interest i in the control sample, ⁱⱼQ_{T} is the frequency for a base type j at position i in the samples collected from a subject having a tumor or suspected to have a tumor,
wherein
$${}_{j}^{i}{Q}{}_{v}^{} = \frac{1}{2} ( {}_{j}^{i}{Q}{}_{T}^{} + {}_{j}^{i}{Q}{}_{N}^{} ) .$$

5. The method of claim 1, wherein the significance score is calculated by the formula:
$${S}_{i} = \frac{1}{2} [ {\sum }_{j = 1}^{4} {}_{j}^{i}{Q}{}_{v}^{} {log}_{2} \frac{{}_{j}^{i}{Q}{}_{v}^{}}{{}_{j}^{i} R} + {\sum }_{j = 1}^{4} {}_{j} p {log}_{2} \frac{{}_{j} p}{{}_{j}^{i} R} ]$$
wherein ⱼp is the background frequency of base j in a reference human genome, wherein
$${}_{j}^{i} R = \frac{1}{2} ( {}_{j}^{i}{Q}{}_{v}^{} + {}_{j} p ) .$$

6. The method of claim 5, wherein the reference human genome is human genome assembly GRCh37 (hg19) or human genome assembly GRCh38(hg38).

7. The method of claim 1, wherein the information score is calculated by the formula:
$${I}_{i} = \frac{1}{2} ( 1 - {D}_{i} ) ( 1 + {S}_{i} ) .$$

8. The method of claim 1, wherein the sequencing results at the position of interest is removed if the information score is higher than a reference threshold.

9. The method of claim 1, wherein the sequencing results at the position of interest is included if the information score is lower than a reference threshold.

10. A system for cancelling noise in sequencing results comprising:
a) at least one device configured to sequence nucleic acid samples comprising a first group of nucleic acid samples collected from one or more control subjects and a second group of nucleic acid samples collected from a subject having a tumor or suspected to have a tumor;
b) a computer-readable program code comprising instructions to execute the following:
i. calculating frequencies for each base type in the first group of nucleic acid samples and frequencies for each base type in the second group of nucleic acid samples at a position of interest in the genome;
ii. calculating a divergence score for position of interest by calculating mutual entropy between the distribution of base type frequencies in the first group of samples and the distribution of base type frequencies in the second group of samples;
iii. calculating a significance score by determining that probability that the distribution of base type frequencies in the first group of samples and the distribution of base type frequencies in the second group of samples represent the same distribution;
iv. calculating an information score based on the divergence score and the significance score, wherein a higher information score indicates that sequencing results at the position of interest is more likely to be noise;
c) a computer-readable program code comprising instructions to execute the following:
i. removing the sequencing results at the position of interest if the information score is higher than a reference threshold; or
ii. including the sequencing results at the position of interest if the information score is lower than a reference threshold.

11. A method for cancelling noise in sequencing results, the method comprising:
(a) determining a ratio of frequencies of each base type in control samples to frequencies of each base type in a reference genome;
(b) determining a ratio of frequencies of each base type in a sample collected from a subject having a tumor or suspected to have a tumor as compared to frequencies of each base type in a reference genome;
(c) determining a score for log of ratios of frequencies of each base type; and
(d) removing the sequencing results if the score has an absolute value that is higher than a reference threshold.

12. The method of claim 11, wherein the log of the ratio of frequencies of each base type in samples collected from the subject having a tumor or suspected to have a tumor is determined by the following formula
$${}_{j}^{i}{w}{}_{T}^{} = ln \frac{{}_{j}^{i}{Q}{}_{T}^{}}{{}_{j} p}$$
wherein ⱼp is the background frequency of a base type j in a reference human genome, and ⁱⱼQ_{T} is the frequency for the base type j at position i in the sample collected from a subject having a tumor or suspected to have a tumor.

13. The method of claim 11, wherein the log of the ratio of frequencies of each base type in control samples is determined by the following formula
$${}_{j}^{i}{w}{}_{N}^{} = ln \frac{{}_{j}^{i}{Q}{}_{N}^{}}{{}_{j} p}$$
wherein ⱼp is the background frequency of a base type j in a reference human genome, and wherein ⁱⱼQ_{N} is the frequency for the base type j at position i in the control samples.

14. The method of claim 11, wherein the score is determined by the following formula:
$${P}_{i} = {\sum }_{j = 1}^{4} {}_{j}^{i}{w}{}_{T}^{} {}_{j}^{i}{w}{}_{N}^{}$$

15. The method of claim 11, wherein the score is determined by the following formula:
$${M}_{i} = {\sum }_{j = 1}^{4} ( {}_{j}^{i}{w}{}_{T}^{} {+}_{j}^{i} {w}_{N} )$$

16-23. (canceled)
